(19) United States
(12) Patent Application Publication
(10) Pub. No.: US 2023/0398108 A1
(43) Pub. Date: Dec. 14, 2023

(54) Pharmaceutical Compositions of a Kinase Inhibitor

(71) Applicant: Exelixis, lnc., Alameda, CA (US)

(72) Inventors: Iswadi LIEJANTO, San Bruno, CA (US); Tzu-Yuan CHEN, San Ramon, CA (US)

(21) Appl. No.: 18/035,355

(22) PCT Filed: Nov. 4, 2021
(86) PCT No.: PCT/US21/57996

§ 371 Date: May 4, 2023

Related U.S. Application Data
(60) Provisional application No. 63/110,124, filed on Nov. 5, 2020.

(51) Int. Cl. A61K 31/47; A61K 9/20; A61K 9/28

(52) U.S. Cl. CPC A61K 31/47; A61K 9/2054; A61K 9/2018; A61K 9/2009; A61K 9/2013; A61K 9/28; A61K 9/2095

(57) ABSTRACT

The present invention relates to pharmaceutical compositions of the c-Met inhibitor, Compound 1. The invention also relates to methods of treating a disease, disorder, or syndrome mediated at least in part by modulating in vivo activity of a protein kinase using the pharmaceutical composition and to processes for making the pharmaceutical compositions.

### CROSS-REFERENCE TO RELATED APPLICATION

This application claims priority to U.S. Provisional Application Ser. No. 63/110,124, the entire contents of which is incorporated by reference herein.

### TECHNICAL FIELD OF THE INVENTION

The present invention relates to pharmaceutical compositions of the free base or pharmaceutically acceptable salts of Compound 1. The invention also relates to pharmaceutical formulations of crystalline salt forms of Compound 1. The invention further relates to methods of treating a disease, disorder, or syndrome mediated at least in part by modulating in vivo activity of a protein kinase by Compound 1 as a pharmaceutical composition.

### BACKGROUND OF THE INVENTION

Human Axl belongs to the Tyro3, Axl, and Mer (TAM) subfamily of receptor tyrosine kinases that includes Mer. TAM kinases are characterized by an extracellular ligand binding domain consisting of two immunoglobulin-like domains and two fibronectin type III domains. Axl is overexpressed in a number of tumor cell types and was initially cloned from patients with chronic myelogenous leukemia. When overexpressed, Axl exhibits transforming potential. Axl signaling is believed to cause tumor growth through activation of proliferative and anti-apoptotic signaling pathways. Axl has been associated with cancers such as lung cancer, myeloid leukemia, uterine cancer, ovarian cancer, gliomas, melanoma, thyroid cancer, renal cell carcinoma, osteosarcoma, gastric cancer, prostate cancer, and breast cancer. The over-expression of Axl results in a poor prognosis for patients with the indicated cancers.

Activation of Mer, like Axl, conveys downstream signaling pathways that cause tumor growth and activation. Mer binds ligands such as the soluble protein Gas-6. Gas-6 binding to Mer induces autophosphorylation of Mer on its intracellular domain, resulting in downstream signal activation. Over-expression of Mer in cancer cells leads to increased metastasis, most likely by generation of soluble Mer extracellular domain protein as a decoy receptor. Tumor cells secrete a soluble form of the extracellular Mer receptor which reduces the ability of soluble Gas-6 ligand to activate Mer on endothelial cells, leading to cancer progression.

A need therefore exists for compounds that inhibit TAM receptor tyrosine kinases such as Axl and Mer for the treatment of selected cancers.

### SUMMARY OF THE INVENTION

The present invention provides pharmaceutical compositions of Compound 1, N-(4-fluorophenyl)-N-(4-((7-methoxy-6-(methylcarbamoyl)quinolin-4-yl)oxy)phenyl)cyclopropane-1,1-dicarboxamide, which has the structure:

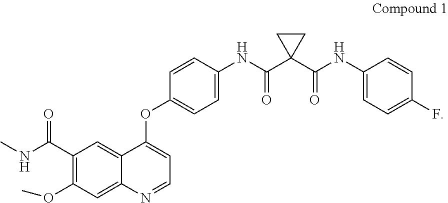 [SMILES: CNC(=O)c1cc2c(Oc3ccc(NC(=O)C4(C(=O)Nc5ccc(F)cc5)CC4)cc3)ccnc2cc1OC]

or pharmaceutically acceptable salts thereof, wherein Compound 1 is a crystalline solid. The formulations disclosed herein were surprisingly found to have improved manufacturability and properties as compared to other forms.

Compound 1 is disclosed in WO 2019/148044, the contents of which is incorporated herein by reference in its entirety. Various crystalline solid forms and crystalline salts of Compound 1 are disclosed in WO 2020/123800, the entire contents of which are incorporated herein by reference.

In one aspect, the pharmaceutical composition is a pharmaceutical composition suitable for oral administration. The pharmaceutical composition comprises:
a. Compound 1 or a pharmaceutically acceptable salt thereof;
b. one or more diluents;
c. one or more binders;
d. one or more disintegrants;
e. one or more glidants;
f. one or more lubricants; and optionally
g. a film coating.

Compound 1 can be present in the pharmaceutical composition as a free base crystalline solid or as a crystalline pharmaceutically acceptable salt. For avoidance of doubt, “Compound 1” includes these crystalline free base forms as well as crystalline salt forms unless otherwise indicated.

In one embodiment of this aspects, Compound 1 is a crystalline solid form characterized as Form A, Form B, Form C, Form D, Form E, Form F, Form G, Form H, Form K, Form O, or Form Q.

In another embodiment of these aspects, Compound 1 is a crystalline HCl salt of Compound 1.

In another embodiment of these aspects, Compound 1 is a crystalline fumaric acid salt of Compound 1, or hydrate or solvate thereof.

In another embodiment, Compound 1 is a crystalline phosphoric acid salt of Compound 1 or hydrate or solvate thereof.

In still another aspect, the invention relates to a method of treating a disease, disorder, or syndrome mediated at least in part by modulating in vivo activity of a protein kinase, comprising administering to a subject in need thereof a pharmaceutical composition of Compound 1 or a pharmaceutically acceptable salt thereof.

In still another aspect, the invention relates to a method of treating cancer, comprising administering to a subject in need thereof a pharmaceutical composition of Compound 1 or a pharmaceutically acceptable salt thereof.

In another aspect, the invention relates to a method for inhibiting a protein kinase, the method comprising contacting the protein kinase with a pharmaceutical composition of Compound 1 as a crystalline form or a crystalline salt form as described herein.

In yet another aspect, the invention relates to a process of preparing a pharmaceutical composition of Compound 1.

### DETAILED DESCRIPTION OF THE INVENTION

### Definitions, Abbreviations and Acronyms

Analytical Techniques

| Abbreviations/Acronyms | Full Name/Description |
|---|---|
| DSC | Differential scanning calorimetry |
| DVS | Dynamic (water) vapor sorption |
| HSM | Hot stage microscopy |
| NMR | Nuclear magnetic resonance spectroscopy |
| OM | Optical microscopy |
| PLM | Polarized light microscopy |
| TGA | Thermogravimetry or Thermogravimetric |
| | analysis |
| XRPD | X-ray powder diffraction |

Experimental techniques

| Abbreviations/Acronyms | Full Name/Description |
|---|---|
| CC | Crash cooling |
| CP | Crash precipitation |
| FC | Fast cooling |
| FE | Fast evaporation |
| RC | Reaction crystallization |
| SC | Slow cooling |
| SE | Slow evaporation |
| VD | Vapor diffusion |
| VS | Vapor stress |

Miscellaneous

| Abbreviations/Acronyms | Full Name/Description |
|---|---|
| ~ | About or approximately |
| API | Active pharmaceutical ingredient |
| B/E | Birefringence and extinction |
| Endo/endo | Endotherm or endothermic |
| eq | Equivalent |
| Exo/exo | Exotherm or exothermic |
| FB | Free base |
| FF | Free form |
| frz | Freezer |
| LIMS | Laboratory Information Management System |
| Max/max | Maximum or maxima |
| Obs | Observation |
| PO | Preferred orientation |
| ppt | Precipitate or precipitation |
| ref | Refrigerator |
| RH | Relative humidity |
| RT | Room temperature |
| Soln/soln | Solution |
| vac | Vacuum |
| wt % | Weight percent |

Solvents

| Abbreviations/Acronyms | Full Name/Description |
|---|---|
| ACN | Acetonitrile |
| AcOH | Acetic acid |
| DCM | Dichloromethane |
| DMSO | Dimethylsulfoxide |
| EtOAc | Ethyl acetate |
| EtOH | Ethanol |
| HFIPA | Hexafluoroisopropanol |
| IPA | Isopropyl alcohol, 2-propanol |
| MEK | Methyl ethyl ketone |
| MeOH | Methanol |
| MTBE | Methyl-tertiary-butyl ether |
| TFE | 2,2,2-Trifluoroethanol |
| THF | Tetrahydrofuran |

As used herein, the following definitions shall apply unless otherwise indicated.

For purposes of this invention, the chemical elements are identified in accordance with the Periodic Table of the Elements, CAS version, Handbook of Chemistry and Physics, 95th Ed. Additionally, general principles of organic chemistry are described in “Organic Chemistry,” 2^{nd} Ed., Thomas Sorrell, University Science Books, Sausalito: 2006, and “March's Advanced Organic Chemistry,” 7th Ed., Ed.: Smith, M. B. and March, J., John Wiley & Sons, New York: 2013, the entire contents of which are hereby incorporated by reference.

As used herein, the term “about”, when referring to a numerical value or range, allows for a degree of variability in the value or range, for example, within 10%, within 5%, within 4%, within 3%, within 2%, within 1%, or within 0.5% of a stated value or of a stated limit of a range. The stated value can be doses, amounts or weight percent of ingredients of a composition or a dosage form.

As used herein, the term “Low/limited/significant hygroscopisity” refers to a material that exhibits <0.5/<2.0/≥2.0 wt % water uptake over a specified RH range.

As used herein, the term “stoichiometric hydrate” refers to crystalline material with a defined water content over an extended RH range. Typical stoichiometric hydrates are hemihydrates, monohydrates, sesquihydrates, dihydrates, etc.

As used herein, the term “variable hydrate” refers to crystalline material with variable water content over an extended RH range, yet with no phase change.

As used herein, a chemical term designated as a “Form” refers to a chemical compound or salt thereof that consists of a single phase.

As used herein, the term “low/limited/intermediate/good/high solubility” refers to a material having a solubility of <1/1-20/20-100/100-200/>200 mg/mL.

As used herein, the term “disordered crystalline” refers to a material that produces XRPD pattern with broad peaks (relative to instrumental peak widths) and/or strong diffuse scattering relative to the peaks. Disordered materials may be:
1) microcrystalline,
2) crystalline with large defect density,
3) mixtures of crystalline and X-ray amorphous phases, or
4) a combination of the above.

As used herein, the term “insufficient signal” means that spectrographic analysis of a sample produced a spectrum or pattern (output) having insufficient signal above the expected background noise.

As used herein, the term “single crystalline phase” refers to an XRPD pattern that is judged to contain evidence of a single crystalline form due to the Bragg peaks being indexed with a single unit cell. Indexing is the process of assigning Miller index labels to each peak in a diffraction pattern. Also, the size and shape of the crystal unit cell is determined during the indexing process.

As used herein, the term “slurry” refers to a suspension prepared by adding enough solids to a given solvent at ambient conditions so that undissolved solids are present. A typical slurry includes agitation (typically by stirring or oscillation), an act that is also referred to as “slurrying,” in a sealed vial at a given temperature for an extended period of time. Typically, the solids are recovered after a given period of time using a method described herein.

As used herein, the term “X-ray amorphous” or “amorphous” refers to a material having diffuse scatter present, but no evidence for Bragg peaks in the XRPD pattern.

As used herein, the term “crystalline” refers to compounds in a solid state having a periodic and repeating three-dimensional internal arrangement of atoms, ions or molecules characteristic of crystals, for example, arranged in fixed geometric patterns or lattices that have rigid long range order. The term crystalline does not necessarily mean that the compound exists as crystals, but that it has this crystal-like internal structural arrangement. Compounds that are crystalline produce an XRPD pattern with sharp peaks (similar to instrumental peak widths) and weak diffuse scattering relative to the peaks.

As used herein, the term “substantially crystalline” refers to a solid material that is predominately arranged in fixed geometric patterns or lattices that have rigid long range order. For example, substantially crystalline materials have more than about 85% crystallinity (e.g., more than about 90% crystallinity, more than about 95% crystallinity, or more than about 99% crystallinity or about 100 crystallinity). It is also noted that the term ‘substantially crystalline’ includes the descriptor ‘crystalline,’ which is defined in the previous paragraph.

“Patient” for the purposes of the present invention includes humans and any other animals, particularly mammals, and other organisms. Thus, the methods are applicable to both human therapy and veterinary applications. In a preferred embodiment, the patient is a mammal, and in a most preferred embodiment the patient is human. Examples of the preferred mammals include mice, rats, other rodents, rabbits, dogs, cats, swine, cattle, sheep, horses, and primates.

“Kinase-dependent diseases or conditions” refer to pathologic conditions that depend on the activity of one or more kinases. Kinases either directly or indirectly participate in the signal transduction pathways of a variety of cellular activities including proliferation, adhesion, migration, differentiation, and invasion. Diseases associated with kinase activities include tumor growth, the pathologic neovascularization that supports solid tumor growth, and associated with other diseases where excessive local vascularization is involved such as ocular diseases (diabetic retinopathy, age-related macular degeneration, and the like) and inflammation (psoriasis, rheumatoid arthritis, and the like).

“Therapeutically effective amount” is an amount of a crystalline form or crystalline salt form of the present invention that, when administered to a patient, ameliorates a symptom of the disease. The amount of a crystalline form or crystalline salt form of the present invention which constitutes a “therapeutically effective amount” will vary depending on the compound, the disease state and its severity, the age of the patient to be treated, and the like. The therapeutically effective amount can be determined routinely by one of ordinary skill in the art having regard to his own knowledge and to this disclosure.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit risk ratio.

As used herein, the phrase “pharmaceutically acceptable excipient” refers to a pharmaceutically-acceptable material, composition, or vehicle, such as a liquid or solid filler, diluent, solvent, or encapsulating material. Excipients are generally safe, non-toxic and neither biologically nor otherwise undesirable and include excipients that are acceptable for veterinary use as well as human pharmaceutical use. In one embodiment, each component is “pharmaceutically acceptable” as defined herein. See, e.g., Remington: The Science and Practice of Pharmacy, 21st ed.; Lippincott Williams & Wilkins: Philadelphia, Pa., 2005; Handbook of ‘Pharmaceutical Excipients, 6th ed.; Rowe et al, Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Pref or mulation and Formulation, 2nd ed.; Gibson Ed.; CRC Press LLC: Boca Raton, Fla., 2009.

As used herein, the term “strength” refers to the weight of Compound 1, as a free base equivalent, in a unit dosage form of a pharmaceutical composition. For example, a tablet comprising 22.20 mg of Compound 1 hemifumarate salt is a tablet of 20 mg dosage strength because 22.20 mg of Compound 1 hemifumarate is equivalent to 20 mg of Compound 1 free base. Similarly, a tablet comprising 44.40 mg of Compound 1 hemifumarate is a tablet of 40 mg strength.

As used herein, the term “concurrently” means at the same time. For example, if two treatment regimens for a single patient are being conducted concurrently, then they are being conducted at the same time. It will be understood that two treatment regimens happening at the same time, does not necessarily mean that actual delivery of two drugs happens at the same time, as each regimen may call for a different dosing schedule and/or different delivery modes.

“Cancer” refers to any physiological condition in mammals characterized by unregulated cell growth; in particular, cellular-proliferative disease states, including, but not limiting to: Cardiac: sarcoma (angiosarcoma, fibrosarcoma, rhabdomyosarcoma, liposarcoma), myxoma, rhabdomyoma, fibroma, lipoma and teratoma; Head and neck: squamous cell carcinomas of the head and neck, laryngeal and hypopharyngeal cancer, nasal cavity and paranasal sinus cancer, nasopharyngeal cancer, salivary gland cancer, oral and orppharyngeal cancer; Lung: bronchogenic carcinoma (squamous cell, undifferentiated small cell, undifferentiated large cell, adenocarcinoma, non-small cell lung cancer), alveolar (bronchiolar) carcinoma, alveolar sarcoma, alveolar soft part sarcoma, bronchial adenoma, sarcoma, lymphoma, chondromatous hamartoma, mesothelioma; Colon: colorectal cancer, adenocarcinoma, gastrointestinal stromal tumors, lymphoma, carcinoids, Turcot Syndrome; Gastrointestinal: gastric cancer, gastroesophageal junction adenocarcinoma, esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Breast: metastatic breast cancer, ductal carcinoma in situ, invasive ductal carcinoma, tubular carcinoma, medullary carcinoma, mucinous carcinoma, lobular carcinoma in situ, triple negative breast cancer; Genitourinary tract: kidney (adenocarcinoma, Wilm's tumor [nephroblastoma], lymphoma, leukemia, renal cell carcinoma, metastatic renal cell carcinoma), bladder and urethra (squamous cell carcinoma, transitional cell carcinoma, adenocarcinoma, urothelial carcinoma), prostate (adenocarcinoma, sarcoma, castrate resistant prostate cancer, bone metastases, bone metastases associated with castrate resistant prostate cancer,), testis (seminoma, teratoma, embryonal carcinoma, teratocarcinoma, choriocarcinoma, sarcoma, interstitial cell carcinoma, fibroma, fibroadenoma, adenomatoid tumors, lipoma), clear cell carcinoma, papillary carcinoma, penile cancer, penile squamous cell carrcinoma; Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma, and giant cell tumors; Thyroid: medullary thyroid cancer, differentiated thyroid cancer, papillary thyroid cancer, follicular thyroid cancer, hurthle cell cancer, and anaplastic thyroid cancer; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma), NF1, neurofibromatosis, plexiform neurofibromas; Gynecological: uterus (endometrial cancer), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia [acute and chronic], acute lymphoblastic leukemia, chronic lymphocytic leukemia, myeloproliferative diseases, multiple myeloma, mvelodysplastic syndrome), myelofibrosis, polycythemia vera, essential thrombocythemia, Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above-identified conditions. In some embodiments, a compound or combination as disclosed herein can be used for the treatment of diseases including HIV, sickle cell disease, graft-versus-host disease, acute graft-versus-host disease, chronic graft-versus-host disease, and sickle cell anemia. In some embodiments, the cancer is clear cell renal cell carcinoma, non-clear cell carcinoma, non-clear cell renal cell carcinoma, salivary gland cancer, penile squamous cell carcinoma, neuroendocrine tumors, adrenocortical carcinoma, or merkel cell carcinoma.

The terms “treating” or “treatment” refer to any indicia of success or amelioration of the progression, severity, and/or duration of a disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being.

The term “enhance” refers to an increase or improvement in the function or activity of a protein or cell after administration or contacting with a combination described herein compared to the protein or cell prior to such administration or contact.

The term “administering” refers to the act of delivering a combination or composition described herein into a subject by such routes as oral, mucosal, topical, suppository, intravenous, parenteral, intraperitoneal, intramuscular, intralesional, intrathecal, intranasal or subcutaneous administration. Parenteral administration includes intravenous, intramuscular, intra-arteriole, intradermal, subcutaneous, intraperitoneal, intraventricular, and intracranial administration. Administration generally occurs after the onset of the disease, disorder, or condition, or its symptoms but, in certain instances, can occur before the onset of the disease, disorder, or condition, or its symptoms (e.g., administration for patients prone to such a disease, disorder, or condition).

The term “coadministration” refers to administration of two or more agents (e.g., a combination described herein and another active agent such as an anti-cancer agent described herein). The timing of coadministration depends in part of the combination and compositions administered and can include administration at the same time, just prior to, or just after the administration of one or more additional therapies, for example cancer therapies such as chemotherapy, hormonal therapy, radiotherapy, or immunotherapy. The compound of the invention can be administered alone or can be coadministered to the patient. Coadministration is meant to include simultaneous or sequential administration of the compound individually or in combination (more than one compound or agent). Thus, the preparations can also be combined, when desired, with other active substances (e.g., to reduce metabolic degradation). The compounds described herein can be used in combination with one another, with other active agents known to be useful in treating cancer.

The term “anti-cancer agent” is used in accordance with its plain ordinary meaning and refers to a composition having anti-neoplastic properties or the ability to inhibit the growth or proliferation of cells. In embodiments, an anti-cancer agent is a chemotherapeutic. In embodiments, an anti-cancer agent is an agent identified herein having utility in methods of treating cancer. In embodiments, an anti-cancer agent is an agent approved by the FDA or similar regulatory agency of a country other than the USA, for treating cancer.

The term “chemotherapeutic” or “chemotherapeutic agent” is used in accordance with its plain ordinary meaning and refers to a chemical composition or compound having anti-neoplastic properties or the ability to inhibit the growth or proliferation of cells. “Chemotherapy” refers to a therapy or regimen that includes administration of a chemotherapeutic or anti-cancer agent described herein.

In general, the nomenclature used in this application is based on naming conventions adopted by the international union of pure and applied chemistry (IUPAC). Chemical structures shown herein were prepared using CHEMDRAW®. Any open valency appearing on a carbon, oxygen, or nitrogen atom in the structures herein indicates the presence of a hydrogen atom.

### Embodiments

### Pharmaceutical Composition

The disclosure is directed to a pharmaceutical composition suitable for oral administration comprising Compound I or a pharmaceutically acceptable salt thereof.

In one aspect, the pharmaceutical composition comprises:
a. Compound 1;
b. one or more diluents;
c. one or more binders;
d. one or more disintegrants;
e. one or more glidants;
f. one or more lubricants; and optionally
g. a film coating.

Compound 1 has the structure

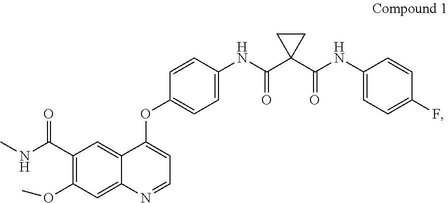 [SMILES: CNC(=O)c1cc2c(Oc3ccc(NC(=O)C4(C(=O)Nc5ccc(F)cc5)CC4)cc3)ccnc2cc1OC]

and is also known as 1-N′-(4-Fluorophenyl)-1-N-[4-[7-methoxy-6-(methylcarbamoyl)quinolin-4-yl]oxyphenyl]cyclopropane-1,1-dicarboxamide, or N′-(4-Fluorophenyl)-N-[4-[7-methoxy-6-(methylcarbamoyl)quinolin-4-yl]oxyphenyl]cyclopropane-1,1-dicarboxamide. As used here. Compound 1 includes crystalline freebase solid forms of Compound 1 as well as crystalline salt forms of Compound 1, or salts, solvates, or hydrates thereof.

Examples of pharmaceutically acceptable diluents, binders, disintegrants, glidants, lubricants, and coatings are described in more detail in references readily available to the skilled practitioner, for instance, in the Handbook of Pharmaceutical Excipients, 7th Ed. R. Rowe, P. Sheskey, and S. Owen, Eds., 2012, Pharmaceutical Press, London England.; and Remington, The Science and Practice of Pharmacy, 21^{st} Ed. P. Gerbino, Ed., Lipincott Williams & Wilkins, Philadelphia, PA.

The diluent may be any diluent known to a person of ordinary skill in the art. In one embodiment, the diluent is an inorganic diluent, polysaccharide, mono- or disaccharide or sugar alcohol. In another embodiment, the diluent comprises lactose, microcrystalline cellulose, starch, corn starch, croscarmellose sodium, or a mixture thereof.

The binder may be any binder known to a person of ordinary skill in the art. Suitable binders comprises sodium carboxymethylcellulose, polyvinyl pyrrolidone (PVP), copovidone, polyvinyl pyrrolidone-vinyl acetate (PVP/VA) copolymer, hydroxypropylcellulose, hydroxypropyl methylcellulose, ethyl cellulose, or a mixture thereof. In another embodiment, the binder is PVP. In another embodiment, the binder is hydroxypropylcellulose.

The disintegrant may be any disintegrant known to a person of ordinary skill in the art. Suitable disintegrants comprises croscarmellose sodium, crospovidone, low-substituted hydroxypropylcellulose, sodium starch glycolate, or a mixture thereof.

The glidant may be any glidant known to a person of ordinary skill in the art. Suitable glidants include starch, corn starch, silicon dioxide, colloidal silicon dioxide, or a mixture thereof. In another embodiment, the glidant is silicon dioxide. In another embodiment, the glidant is colloidal silicon dioxide.

The lubricant may be any lubricant known to a person of ordinary skill in the art. In another embodiment, the lubricant is stearic acid or magnesium stearate.

In these and other embodiments, the film coating may be any film coating known to a person of ordinary skill in the art. Such coatings are widely commercially available, such as coatings that contain as ingredients

The terms “film coating” and “film-coated” as used herein relates to a mixture of pharmaceutically acceptable excipients which are typically applied to a compressed tablet, beads, granules, or particles of active ingredient that are compressed into tablets. It is understood that the coating chosen must be compatible with the active agent. It is further understood that a person skilled in the art will know how to manipulate the coating to achieve disintegration in the stomach by choosing the excipients which make up the coating, its type, and/or its thickness.

Suitable polymers for film-coating according to the present invention are soluble at pH of from about 1.2 to about 5, such as for example hydroxypropylmethylcellulose (HPMC) alone and/or in combination with hydroxypropylcellulose (HPC), carboxymethylcellulose, methylcellulose, ethylcellulose, acrylic resins, and polyvinylpyrrolidone and gelatin or other commercially available film-coating preparations such as Dri-Klear® (Crompton & Knowles Corp., Mahwah, N.J.) or Opadry® (Colorcon, West Point Pa.).

In another embodiment, the film coating is comprised of a commercial film-coating product designed for aqueous film coating containing the water-soluble, film-forming resin, hydroxypropyl methylcellulose and polyethylene glycol (or other suitable plasticizing agents such as propylene glycol or glycerine) and optionally containing titanium dioxide (or other colorant or opacifying agent). Such a product is commercially available under the trade name Opadry® II Blue (Colorcon, West Point, Pa.).

A suitable blend for a coating may comprise 0 to about 20% w/w titanium dioxide or colorant, about 5 to about 95% w/w hydroxypropyl methylcellulose, and 0 to about 25% w/w polyethylene glycol. In one embodiment, the coating comprises 10.5% non-water additives, of which 7.5% is Opadry®, in relation to the total weight of the coating.

The coating may further comprise flavoring agents, taste-masking agents and salivating agents as defined hereinabove, in small amounts such as for example 0.1 to 1.0% (w/w), preferably 0.1 to 0.4% based on the weight of the total blend for coating. The preferred flavoring and/or taste-masking agent may be selected from the group of agents as defined hereinabove.

The amount of coating deposited on the tablet is typically in the range of from about 1.0% to about 6.0% weight gain, preferably from 2.0% to 5.0% weight gain, which means the weight gain of the tablet upon coating relative to the weight of the uncoated tablet.

In one embodiment, the pharmaceutical composition comprises:
a. Compound 1 or a pharmaceutically acceptable salt thereof;
b. one or more diluents selected from the group consisting of an inorganic diluent, polysaccharide, mono- or disaccharide, sugar alcohol, and a mixture thereof;
c. one or more binders selected from the group consisting of sodium carboxymethylcellulose, polyvinyl pyrrolidone (PVP), copovidone, polyvinyl pyrrolidone-vinyl acetate (PVP/VA) copolymer, hydroxypropylcellulose, hydroxypropyl methylcellulose, ethyl cellulose, and a mixture thereof;
d. one or more disintegrants selected from the group consisting of croscarmellose sodium, crospovidone, low-substituted hydroxypropylcellulose, starch, sodium starch glycolate, and a mixture thereof;
e. one or more glidants;
f. one or more lubricants; and optionally
g. a film coating.

In one embodiment, the pharmaceutical composition comprises:
a. Compound 1 or a pharmaceutically acceptable salt thereof;
b. one or more diluents selected from the group consisting of an inorganic diluent, polysaccharide, mono- or disaccharide and sugar alcohol;
c. one or more binders selected from the group consisting of sodium carboxymethylcellulose, polyvinyl pyrrolidone (PVP), copovidone, polyvinyl pyrrolidone-vinyl acetate (PVP/VA) copolymer, hydroxypropylcellulose, hydroxypropyl methylcellulose and ethyl cellulose;
d. one or more disintegrants selected from the group consisting of croscarmellose sodium, crospovidone, low-substituted hydroxypropylcellulose, and sodium starch glycolate;
e. one or more glidants;
f. one or more lubricants; and optionally
g. a film coating.

In one embodiment, the pharmaceutical composition comprises:
a. Compound 1 or a pharmaceutically acceptable salt thereof;
b. one or more diluents selected from the group consisting of an inorganic diluent, polysaccharide, mono- or disaccharide and sugar alcohol;
c. one or more binders selected from the group consisting of sodium carboxymethylcellulose, polyvinyl pyrrolidone (PVP), copovidone, polyvinyl pyrrolidone-vinyl acetate (PVPNA) copolymer, hydroxypropylcellulose, hydroxypropyl methylcellulose and ethyl cellulose;
d. one or more disintegrants selected from the group consisting of croscarmellose sodium, crospovidone, low-substituted hydroxypropylcellulose, and sodium starch glycolate;
e. silicon dioxide;
f. one or more lubricants; and optionally
g. a film coating.

In one embodiment, the pharmaceutical composition comprises:
a. Compound 1 or a pharmaceutically acceptable salt thereof;
b. one or more diluents selected from the group consisting of an inorganic diluent, polysaccharide, mono- or disaccharide and sugar alcohol;
c. one or more binders selected from the group consisting of sodium carboxymethylcellulose, polyvinyl pyrrolidone (PVP), copovidone, polyvinyl pyrrolidone-vinyl acetate (PVPNA) copolymer, hydroxypropylcellulose, hydroxypropyl methylcellulose and ethyl cellulose;
d. one or more disintegrants selected from the group consisting of croscarmellose sodium, crospovidone, low-substituted hydroxypropylcellulose, and sodium starch glycolate;
e. silicon dioxide;
f. stearic acid or magnesium stearate; and optionally
g. a film coating.

In certain embodiments, the pharmaceutical compositions of the disclosure can be compacted into a unit dose form, such as a tablet, or caplet, or added to unit dose form, e.g., a capsule. In a further embodiment, pharmaceutical compositions of the disclosure can be formulated for administration as a powder or suspension. A pharmaceutical formulation of the disclosure which comprises a powder can, for example, be sprinkled on or mixed with a semi-solid carrier such as apple sauce or another food item for administration to a subject. The powder can also, for example, be added to a liquid carrier suitable for administration to subjects, such as a solution of about 2% w/V hydroxypropyl cellulose and about 0.1% w/V polysorbate 80 in water or about 0.2% hydroxypropylcellulose, and 0.1% Tween 80 in water, to form a suspension.

In one embodiment, the dosage form of the disclosure comprises a tablet, containing Compound 1 or a pharmaceutically acceptable salt thereof, at about 5 mg to about 200 mg (free base equivalent), about 10 mg to about 150 mg (free base equivalent), about 15 mg to about 120 mg (free base equivalent), or about 20 mg to about 100 mg (free base equivalent).

In one embodiment, the dosage form of the disclosure comprises a capsule, containing Compound 1 or a pharmaceutically acceptable salt thereof, at about 5 mg to about 200 mg (free base equivalent), about 10 mg to about 150 mg (free base equivalent), about 15 mg to about 120 mg (free base equivalent), or about 20 mg to about 100 mg (free base equivalent).

In one embodiment, the dosage form of the disclosure comprises a tablet, containing Compound 1 or a pharmaceutically acceptable salt thereof, for example, at about (free base equivalent) 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg. In one embodiment, the dosage form of the disclosure comprises a tablet, containing Compound 1 or a pharmaceutically acceptable salt thereof, at about (free base equivalent) 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, or 200 mg. In another embodiment, the dosage form of the disclosure comprises a capsule, for example at about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg strengths. In one embodiment, the dosage form of the disclosure comprises a capsule, containing Compound 1 or a pharmaceutically acceptable salt thereof, at about (free base equivalent) 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, or 200 mg. In a further embodiment, the dosage form of the disclosure is a tablet comprising Compound 1 or a pharmaceutically acceptable salt thereof, for example at about 20 mg, 40 mg, 60 mg, 80 mg or 100 mg strengths. In a further embodiment, the dosage form of the disclosure is a tablet comprising Compound 1 or a pharmaceutically acceptable salt thereof, for example at about 20 mg, 40 mg, 60 mg, 80 mg, 100 mg, or 120 mg strengths. In another embodiment, the dosage form of the disclosure is a capsule comprising Compound 1 or a pharmaceutically acceptable salt thereof, for example at about 20 mg, 40 mg, 60 mg, 80 mg or 100 mg strengths. In a further embodiment, the dosage form of the disclosure is a capsule comprising Compound 1 or a pharmaceutically acceptable salt thereof, for example at about 20 mg, 40 mg, 60 mg, 80 mg, 100 mg, or 120 mg strengths.

Suitable techniques for formulating pharmaceutical compositions of the disclosure into tablets are well-known in the art, and can comprise mixing the active ingredient and stabilizing polymer with one or more pharmaceutically acceptable tableting excipients and compressing the mixture into a tablet, for example with a tableting press. The amount and nature of the tableting excipients used can be readily chosen based on the desired characteristics of the tablet, such as size, hardness, friability and the like. Tablets comprising pharmaceutical compositions of the disclosure can also be coated, for example with film coatings such as the Opadry® coatings available from Colorcon (West Point Pa), or with enteric coatings designed to prevent dissolution of the tablets until the transit the stomach and/or upper intestine. Suitable tablet coatings and methods for applying them are well-known in the art.

Suitable techniques for formulating pharmaceutical compositions of the disclosure into capsules are also well-known in the art, and can comprise mixing the active ingredient and stabilizing polymer with one or more pharmaceutically acceptable capsule excipients and filling the mixture into a capsule. In one embodiment, a pharmaceutical formulation of the disclosure (with or without additional excipients) can be filled into a capsule, such as a hard gelatin capsule. The hard gelatin capsule can be of any appropriate size, for example size ‘0’, ‘0EL’, ‘3’, ‘4’ and the like. For example, in one embodiment a capsule of the disclosure having a dosage strength of 20 mg of Compound 1 can be filled into a hard gelatin capsule of size 4, where the target capsule fill weight can comprise 100 mg. In another embodiment, a capsule of the disclosure having a dosage strength of 100 mg of the active ingredient can be filled into a hard gelatin capsule of size 0el, where the target capsule fill weight can comprise 400 mg.

In one embodiment of the pharmaceutical composition, Compound 1 can be present in at least about 1 percent to about 99 percent by weight (w/w). In another embodiment, Compound 1 can be present in at least about 10 percent to about 90 percent by weight (w/w). In another embodiment, Compound 1 can be present in at least about 20 percent to about 70 percent by weight (w/w). In another embodiment, Compound 1 can be present in at least about 10 percent to about 50 percent by weight (w/w). In another embodiment, Compound 1 can be present in at least about 20 percent to about 40 percent by weight (w/w). In another embodiment, Compound 1 can be present in at least about 25 percent to about 35 percent by weight (w/w). In another embodiment, Compound 1 can be present in the pharmaceutical composition in at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, 99.5%, 99.9% or 100% weight/weight (w/w).

In some embodiments, the pharmaceutical compositions of the disclosure are stable when subject to predetermined conditions for predetermined times. For example, pharmaceutical formulations of the disclosure can be stored at various predetermined temperatures and relative humidities for defined or predetermined time periods, for example in an open or closed container. In some embodiments, pharmaceutical compositions of the disclosure are stable upon storage at about 5, 25, 30, 37, 40 or 45 Celsius and about 0%, 5%, 10%, 15%, 20%, 25%, 30%, 35%, 40%, 45%, 50%, 55%, 60%, 65%, 70%, 75%, 80%, 85%, 90%, 95% or 100% relative humidity for a period of at least about 0.5, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 20, 25, 30, 35, 40, 45, 48, 50, 51, 52, 53, 55 or 60 hours; 1 week, 2 weeks. 3 weeks or 4 week; 1 month, 2 months, 3 months, 4 months, 5 months, 6 months, or 12 months.

In certain embodiments, the pharmaceutical compositions of the disclosure are stable upon storage in an open or closed container at: about 30 degrees Celsius and about 90 percent relative humidity for a period of at least about 20 hours; about 40 degrees Celsius and about 60 percent relative humidity for a period of at least about one week, two weeks or three weeks; about 40 degrees Celsius and about 75 percent relative humidity for a period of at least about one week, two weeks or three weeks; about 25 degrees Celsius and about 60 percent relative humidity for a period of at least about one month; about 40 degrees Celsius and about 75 percent relative humidity for a period of at least one month; about 25 degrees Celsius and about 75 percent relative humidity for a period of at least about 3 months; or 5 degrees Celsius at any relative humidity for a period of at least about three months. In some embodiments, “storage in an open container” means that the container was opened twice a day for a given period of time, for example up to four weeks, but was otherwise left closed.

In another embodiment, the pharmaceutical composition comprises Compound 1 is stable upon storage in an open or closed container at: about 30 degrees Celsius and about 90 percent relative humidity for a period of at least about 20 hours; about 40 degrees Celsius and about 60 percent relative humidity for a period of at least about one week, two weeks or three weeks; about 4 degrees Celsius and about 75 percent relative humidity for a period of at least about one week, two weeks or three weeks; about 25 degrees Celsius and about 60 percent relative humidity for a period of at least about one month; about 40 degrees Celsius and about 75 percent relative humidity for a period of at least one month; about 25 degrees Celsius and about 75 percent relative humidity for a period of at least about 3 months; or 5 degrees Celsius at any relative humidity for a period of at least about three months.

In another embodiment, the pharmaceutical composition comprises Compound 1 is stable upon storage in an open or closed container at: about 25 degrees Celsius and about 60 percent relative humidity for a period of at least about 1 month, 2 months, 3 months, 4 months, 5 months, 6 months, 7 months, 8 months, 9 months, 10 months, 11 months, or 12 months. In another embodiment, the pharmaceutical composition comprises Compound 1 is stable upon storage in an open or closed container at about 25 degrees Celsius and about 60 percent relative humidity for at least 1 month, 2 months, or 3 months with a total impurity of less than 0.1%. In another embodiment, the pharmaceutical composition comprises Compound 1 is stable upon storage in an open or closed container at about 25 degrees Celsius and about 60 percent relative humidity for at least 6 months with a total impurity of less than 0.5%. In another embodiment, the pharmaceutical composition comprises Compound 1 is stable upon storage in an open or closed container at about 25 degrees Celsius and about 60 percent relative humidity for at least 12 months with a total impurity of less than 0.5%.

In another embodiment, the pharmaceutical composition comprises Compound 1 is stable upon storage in an open or closed container at: about 40 degrees Celsius and about 75 percent relative humidity for a period of at least about 1 month, 2 months, 3 months, 4 months, 5 months, 6 months, 7 months, 8 months, 9 months, 10 months, 11 months, or 12 months. In another embodiment, the pharmaceutical composition comprises Compound 1 is stable upon storage in an open or closed container at about 40 degrees Celsius and about 75 percent relative humidity for at least 1 month, 2 months, or 3 months with a total impurity of less than 0.1%. In another embodiment, the pharmaceutical composition comprises Compound 1 is stable upon storage in an open or closed container at about 40 degrees Celsius and about 75 percent relative humidity for at least 6 months with a total impurity of less than 0.5%.

Stability of the pharmaceutical compositions of the disclosure can also be measured by testing other physical characteristics, for example by testing the dissolution of the pharmaceutical composition at the end of a predetermined time period after it has been subjected to predetermined conditions of, for example, temperature and relative humidity for predetermined periods of time. Suitable methods for measuring the dissolution profile of the present pharmaceutical compositions are known in the art. Exemplary methods for measuring the dissolution profile of the present pharmaceutical compositions are either a basket dissolution test or paddle dissolution test, for example in simulated gastric fluid.

In one embodiment, the pharmaceutical composition comprising Compound 1 exhibits greater than 25%, 30%, 40%, or 50% dissolution at 5 minutes after storage in an open or closed container at about 25 degrees Celsius and about 60 percent relative humidity for a period of at least about 0 month, 1 month, 2 months, 3 months, 4 months, 5 months, 6 months, 7 months, 8 months, 9 months, 10 months, 11 months, or 12 months. In another embodiment, the pharmaceutical composition comprising Compound 1 exhibits greater than 50% dissolution at 5 minutes after storage in an open or closed container at about 25 degrees Celsius and about 60 percent relative humidity for a period of at least about 1 month, 2 months, 3 months, 6 months, 7 months, or 12 months. In another embodiment, the pharmaceutical composition comprising Compound 1 exhibits greater than 70% dissolution at 10 minutes after storage in an open or closed container at about 25 degrees Celsius and about 60 percent relative humidity for a period of at least about 0 month, 1 month, 2 months, 3 months, 4 months, 5 months, 6 months. 7 months, 8 months, 9 months, 10 months, 11 months, or 12 months. In another embodiment, the pharmaceutical composition comprising Compound 1 exhibits greater than 90% dissolution at 45 minutes after storage in an open or closed container at about 25 degrees Celsius and about 60 percent relative humidity for a period of at least about 0 month, 1 month, 2 months, 3 months, 4 months, 5 months, 6 months, 7 months, 8 months, 9 months, 10 months, 11 months, or 12 months. In another embodiment, the pharmaceutical composition comprising Compound 1 exhibits greater than 95% dissolution at 75 minutes after storage in an open or closed container at about 25 degrees Celsius and about 60 percent relative humidity for a period of at least about 0 month, 1 month, 2 months, 3 months, 4 months, 5 months, 6 months, 7 months, 8 months, 9 months, 10 months, 11 months, or 12 months.

In another embodiment, the pharmaceutical composition comprising Compound 1 exhibits greater than 25%, 30%, 40%, or 50% dissolution at 5 minutes after storage in an open or closed container at about 40 degrees Celsius and about 75 percent relative humidity for a period of at least about 0 month, 1 month, 2 months, 3 months, 4 months, 5 months, or 6 months. In another embodiment, the pharmaceutical composition comprising Compound 1 exhibits greater than 40%, 50%. 60%, or 70% dissolution at 10 minutes after storage in an open or closed container at about 40 degrees Celsius and about 75 percent relative humidity for a period of at least about 0 month, 1 month, 2 months, 3 months, 4 months, 5 months, or 6 months. In another embodiment, the pharmaceutical composition comprising Compound 1 exhibits greater than 70%, 80%, or 90% dissolution at 45 minutes after storage in an open or closed container at about 40 degrees Celsius and about 75 percent relative humidity for a period of at least about 0 month, 1 month, 2 months, 3 months, 4 months, 5 months, or 6 months. In another embodiment, the pharmaceutical composition comprising Compound 1 exhibits greater than 90% or 95% dissolution at 75 minutes after storage in an open or closed container at about 40 degrees Celsius and about 75 percent relative humidity for a period of at least about 0 month, 1 month, 2 months, 3 months, 4 months, 5 months, or 6 months.

In certain embodiments, pharmaceutical compositions of the disclosure comprise such as tablets, capsules, sachets, powders, suspensions, suppositories and the like. In such dosage forms of the disclosure, the amount of active ingredient comprising the dosage form can be any suitable amount, for example about 0.5 mg, 1 mg, 1.5 mg, 2 mg, 2.5 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 96 mg, 97 mg, 98 mg, 99 mg or 100 mg per unit dosage form. In certain embodiments, dosage forms of the disclosure comprise about 25 mg, 50 mg, 75 mg, 80 mg or 100 mg of the active ingredient per dosage form, for example of Compound 1.

Although exemplary amounts or ranges for Compound 1 and other pharmaceutical composition ingredients are given, pharmaceutical compositions of the disclosure can comprise any amount of these components suitable for the purposes of obtaining the desirable pharmacologic and stability properties as described herein. In addition to these other pharmaceutically acceptable ingredients may be added to the pharmaceutical compositions, for example adjuvants, antioxidants, buffers, coloring agents, compression aids, emulsifiers, emollients, encapsulating materials, fillers, flavoring agents, granulating agents, metal chelators, osmo-regulators, pH adjustors, preservatives, solubilizers, sorbents, stabilizers, sweeteners, surfactants, suspending agents, thickening agents, or viscosity regulators.

In one embodiment, the pharmaceutical composition is a tablet pharmaceutical composition suitable for oral administration.

In a further embodiment, the tablet pharmaceutical composition comprises:
a. Compound 1;
b. Microcrystalline cellulose;
c. Lactose;
d. Hydroxypropyl cellulose;
e. Croscarmellose sodium;
f. Silicon dioxide; and
g. Stearic acid or magnesium stearate; and optionally
h. a film coating.

In one embodiment, the tablet pharmaceutical composition comprises:
a. Compound 1;
b. Microcrystalline cellulose
c. Anhydrous lactose
d. Hydroxypropyl cellulose;
e. Croscarmellose sodium
f. Colloidal silicon dioxide; and
g. Stearic acid or magnesium stearate; and optionally
h. a film coating.

The tablet pharmaceutical compositions of Compound 1 can be described in terms of the weight percent (“by weight”) of each ingredient that is present in a dosage form, wherein the sum of the weight percents does not exceed 100 percent.

Thus, in one embodiment, the tablet pharmaceutical composition comprises:
a. about 20 percent to about 40 percent by weight of Compound 1;
b. about 35 percent to about 45 percent by weight of microcrystalline cellulose;
c. about 15 to about 25 percent by weight of lactose;
d. about 2 to about 8 percent by weight of hydroxypropyl cellulose;
e. about 4 to about 8 percent by weight of croscarmellose sodium;
f. about 0.1 to about 0.5 percent by weight of silicon dioxide; and
g. about 0.5 to about 3.5 percent by weight magnesium stearate; and optionally
h. a film coating.

In one embodiment, the tablet pharmaceutical composition comprises:
a. about 20 percent to about 40 percent by weight of Compound 1;
b. about 35 percent to about 45 percent by weight of microcrystalline cellulose;
c. about 15 to about 25 percent by weight of anhydrous lactose;
d. about 2 to about 8 percent by weight of hydroxypropyl cellulose;
e. about 4 to about 8 percent by weight of croscarmellose sodium;
f. about 0.1 to about 0.5 percent by weight of colloidal silicon dioxide; and
g. about 0.5 to about 3.5 percent by weight magnesium stearate; and optionally
h. a film coating.

In one embodiment, the tablet pharmaceutical composition comprises:
a. about 25 percent to about 35 percent by weight of Compound 1;
b. about 37 percent to about 43 percent by weight of microcrystalline cellulose;
c. about 18 to about 22 percent by weight of anhydrous lactose;
d. about 2 to about 6 percent by weight of hydroxypropyl cellulose;
e. about 5 to about 7 percent by weight of croscarmellose sodium;
f. about 0.2 to about 0.4 percent by weight of colloidal silicon dioxide; and
g. about 0.5 to about 3.5 percent by weight magnesium stearate; and optionally
h. a film coating.

In one embodiment, the tablet pharmaceutical composition comprises:
a. about 25 percent to about 35 percent by weight of Compound 1;
b. about 37 percent to about 43 percent by weight of microcrystalline cellulose;
c. about 18 to about 22 percent by weight of anhydrous lactose;
d. about 2 to about 4 percent by weight of hydroxypropyl cellulose;
e. about 5 to about 7 percent by weight of croscarmellose sodium;
f. about 0.2 to about 0.4 percent by weight of colloidal silicon dioxide; and
g. about 0.5 to about 1.5 percent by weight magnesium stearate; and optionally
h. a film coating.

Thus, in another embodiment, the tablet pharmaceutical composition comprises:
a. about 20 percent to about 40 percent by weight of Compound 1;
b. about 35 percent to about 45 percent by weight of microcrystalline cellulose;
c. about 15 to about 25 percent by weight of lactose;
d. about 2 to about 8 percent by weight of hydroxypropyl cellulose;
e. about 2 to about 8 percent by weight of croscarmellose sodium;
f. about 0.1 to about 0.5 percent by weight of silicon dioxide; and
g. about 1 to about 5 percent by weight of stearic acid; and optionally
h. a film coating.

Thus, in another embodiment, the tablet pharmaceutical composition comprises:
a. about 20 percent to about 40 percent by weight of Compound 1;
b. about 35 percent to about 45 percent by weight of microcrystalline cellulose;
c. about 15 to about 25 percent by weight of anhydrous lactose;
d. about 2 to about 8 percent by weight of hydroxypropyl cellulose;
e. About 2 to about 8 percent by weight of croscarmellose sodium;
f. about 0.1 to about 0.5 percent by weight of colloidal silicon dioxide; and
g. about 1 to about 5 percent by weight of stearic acid; and optionally
h. a film coating.

In one embodiment, the tablet pharmaceutical composition comprises:
a. about 25 percent to about 35 percent by weight of Compound 1;
b. about 35 percent to about 40 percent by weight of microcrystalline cellulose;
c. about 16 to about 22 percent by weight of anhydrous lactose;
d. about 3 to about 7 percent by weight of hydroxypropyl cellulose;
e. about 3 to about 7 percent by weight of croscarmellose sodium;
f. about 0.1 to about 0.5 percent by weight of colloidal silicon dioxide; and
g. about 0.5 to about 3.5 percent by weight stearic acid; and optionally
h. a film coating.

In one embodiment, the tablet pharmaceutical composition comprises:
a. about 25 percent to about 35 percent by weight of Compound 1;
b. about 35 percent to about 40 percent by weight of microcrystalline cellulose;
c. about 16 to about 22 percent by weight of anhydrous lactose;
d. about 3 to about 7 percent by weight of hydroxypropyl cellulose;
e. about 3 to about 7 percent by weight of croscarmellose sodium;
f. about 0.1 to about 0.5 percent by weight of colloidal silicon dioxide; and
g. about 1.5 to about 3.5 percent by weight stearic acid; and optionally
h. a film coating.

In another embodiment, the tablet pharmaceutical composition comprises about 10 to about 150 mg Compound 1 (free base equivalent) and pharmaceutically acceptable excipients selected from the group consisting of one or more diluents, one or more binders, one or more disintegrants, one or more glidants, one or more lubricants, and optionally a film coating.

In another embodiment, the tablet pharmaceutical composition comprises about 10 to about 100 mg Compound 1 and pharmaceutically acceptable excipients selected from the group consisting of one or more diluents, one or more binders, one or more disintegrants, one or more glidants, one or more lubricants, and optionally a film coating.

In another embodiment, the tablet pharmaceutical composition comprises about 10 to about 90 mg Compound 1; microcrystalline cellulose; lactose; hydroxypropyl cellulose; croscarmellose sodium; silicon dioxide; and stearic acid or magnesium stearate; and optionally a film coating.

In another embodiment, the tablet pharmaceutical composition comprises about 5 mg, 10 mg, 15 mg, 20 mg, 25 mg, 30 mg, 35 mg, 40 mg, 45 mg, 50, 55 mg, 60 mg, 65 mg, 70 mg, 75 mg, 80 mg, 85 mg, 90 mg, 95 mg, 100 mg, 110 mg, 120 mg, 130 mg, 140 mg, or 150 mg of Compound 1 (free base equivalent); microcrystalline cellulose; lactose; hydroxypropyl cellulose; croscarmellose sodium; silicon dioxide; and stearic acid or magnesium stearate; and optionally a film coating

In another embodiment, the tablet pharmaceutical composition comprises about 5 mg, 10 mg, 15 mg, 20 mg, 25 mg, 30 mg, 35 mg, 40 mg, 45 mg, 50, 55 mg, 60 mg, 65 mg, 70 mg, 75 mg, 80 mg, 85 mg, 90 mg, 95 mg, or 100 mg of Compound 1; microcrystalline cellulose; lactose; hydroxypropyl cellulose; croscarmellose sodium; silicon dioxide; and stearic acid or magnesium stearate; and optionally a film coating.

In one embodiment, the tablet pharmaceutical composition comprises:
a. 15-150 mg of Compound 1 (free base equivalent);
b. microcrystalline cellulose;
c. lactose;
d. hydroxypropyl cellulose;
e. croscarmellose sodium;
f. silicon dioxide; and
g. magnesium stearate or stearic acid; and optionally
h. a film coating.

In one embodiment, the tablet pharmaceutical composition comprises:
a. 15-150 mg of Compound 1 (free base equivalent);
b. microcrystalline cellulose;
c. anhydrous lactose;
d. hydroxypropyl cellulose;
e. croscarmellose sodium;
f. colloidal silicon dioxide; and
g. magnesium stearate or stearic acid; and optionally
h. a film coating.

In one embodiment, the tablet pharmaceutical composition comprises:
a. 15-100 mg of Compound 1;
b. microcrystalline cellulose;
c. lactose;
d. hydroxypropyl cellulose;
e. croscarmellose sodium;
f. silicon dioxide; and
g. magnesium stearate or stearic acid; and optionally
h. a film coating.

In one embodiment, the tablet pharmaceutical composition comprises:
a. 15-100 mg of Compound 1;
b. microcrystalline cellulose;
c. anhydrous lactose;
d. hydroxypropyl cellulose;
e. croscarmellose sodium;
f. colloidal silicon dioxide; and
g. magnesium stearate or stearic acid; and optionally
h. a film coating.

In one embodiment, the tablet pharmaceutical composition comprises:
a. about 20 mg of Compound 1;
b. microcrystalline cellulose;
c. anhydrous lactose;
d. hydroxypropyl cellulose;
e. croscarmellose sodium;
f. colloidal silicon dioxide; and
g. magnesium stearate or stearic acid; and optionally
h. a film coating.

In one embodiment, the tablet pharmaceutical composition comprises:
a. about 40 mg of Compound 1;
b. microcrystalline cellulose;
c. anhydrous lactose;
d. hydroxypropyl cellulose;
e. croscarmellose sodium;
f. colloidal silicon dioxide; and
g. magnesium stearate or stearic acid; and optionally
h. a film coating.

In one embodiment, the tablet pharmaceutical composition comprises:
a. about 60 mg of Compound 1;
b. microcrystalline cellulose;
c. anhydrous lactose;
d. hydroxypropyl cellulose;
e. croscarmellose sodium;
f. colloidal silicon dioxide; and
g. magnesium stearate or stearic acid; and optionally
h. a film coating.

In one embodiment, the tablet pharmaceutical composition comprises:
a. about 80 mg of Compound 1;
b. microcrystalline cellulose;
c. anhydrous lactose;
d. hydroxypropyl cellulose;
e. croscarmellose sodium;
f. colloidal silicon dioxide; and
g. magnesium stearate or stearic acid; and optionally
h. a film coating.

In one embodiment, the tablet pharmaceutical composition comprises:
a. about 100 mg of Compound 1;
b. microcrystalline cellulose;
c. anhydrous lactose;
d. hydroxypropyl cellulose;
e. croscarmellose sodium;
f. colloidal silicon dioxide; and
g. magnesium stearate or stearic acid; and optionally
h. a film coating.

In one embodiment, the tablet pharmaceutical composition comprises:
i. about 120 mg of Compound 1;
j. microcrystalline cellulose;
k. anhydrous lactose;
l. hydroxypropyl cellulose;
m. croscarmellose sodium;
n. colloidal silicon dioxide; and
o. magnesium stearate or stearic acid; and optionally
p. a film coating.

In one embodiment, the tablet pharmaceutical composition comprises:
a. about 18 to 23 mg of Compound 1;
b. about 30 to 35 mg of microcrystalline cellulose;
c. about 15 to 18 mg anhydrous lactose;
d. about 1.5 to 4.5 mg hydroxypropyl cellulose;
e. about 4 to 6 mg of croscarmellose sodium;
f. about 0.1 to 0.3 mg colloidal silicon dioxide; and
g. about 0.5 to 0.7 mg magnesium stearate; and optionally
h. about 2 to 6 mg of a film coating.

In one embodiment, the tablet pharmaceutical composition comprises:
a. about 20 mg of Compound 1;
b. about 30 to 35 mg of microcrystalline cellulose;
c. about 15 to 18 mg anhydrous lactose;
d. about 1.5 to 4.5 mg hydroxypropyl cellulose;
e. about 4 to 6 mg of croscarmellose sodium;
f. about 0.1 to 0.3 mg colloidal silicon dioxide; and
g. about 0.5 to 0.7 mg magnesium stearate; and optionally
h. about 2 to 6 mg of a film coating.

In one embodiment, the tablet pharmaceutical composition comprises:
a. about 35 to 45 mg of Compound 1;
b. about 60 to 70 mg of microcrystalline cellulose;
c. about 30 to 40 mg lactose;
d. about 2 to 10 mg hydroxypropyl cellulose;
e. about 8 to 12 mg of croscarmellose sodium;
f. about 0.2 to 0.6 mg silicon dioxide; and
g. about 1 to 1.5 mg magnesium stearate; and optionally
h. about 4 to 12 mg of a film coating.

In one embodiment, the tablet pharmaceutical composition comprises:
a. about 40 mg of Compound 1;
b. about 60 to 70 mg of microcrystalline cellulose;
c. about 30 to 40 mg lactose;
d. about 2 to 10 mg hydroxypropyl cellulose;
e. about 8 to 12 mg of croscarmellose sodium;
f. about 0.2 to 0.6 mg silicon dioxide; and
g. about 1 to 1.5 mg magnesium stearate; and optionally
h. about 4 to 12 mg of a film coating.

In one embodiment, the tablet pharmaceutical composition comprises:
a. about 55 to 65 mg of Compound 1;
b. about 90 to 110 mg of microcrystalline cellulose;
c. about 40 to 60 mg anhydrous lactose;
d. about 4 to 14 mg hydroxypropyl cellulose;
e. about 12 to 20 mg of croscarmellose sodium;
f. about 0.3 to 1 mg colloidal silicon dioxide; and
g. about 1.5 to 2.5 mg magnesium stearate; and optionally
h. about 6 to 18 mg of a film coating.

In one embodiment, the tablet pharmaceutical composition comprises:
a. about 60 mg of Compound 1;
b. about 90 to 110 mg of microcrystalline cellulose;
c. about 40 to 60 mg anhydrous lactose;
d. about 4 to 14 mg hydroxypropyl cellulose;
e. about 12 to 20 mg of croscarmellose sodium;
f. about 0.3 to 1 mg colloidal silicon dioxide; and
g. about 1.5 to 2.5 mg magnesium stearate; and optionally
h. about 6 to 18 mg of a film coating.

In one embodiment, the tablet pharmaceutical composition comprises:
a. about 70 to 90 mg of Compound 1;
b. about 120 to 150 mg of microcrystalline cellulose;
c. about 60 to 80 mg anhydrous lactose;
d. about 6 to 18 mg hydroxypropyl cellulose;
e. about 15 to 25 mg of croscarmellose sodium;
f. about 0.4 to 1.5 mg colloidal silicon dioxide; and
g. about 2 to 3 mg magnesium stearate; and optionally
h. about 8 to 26 mg of a film coating.

In one embodiment, the tablet pharmaceutical composition comprises:
a. about 80 mg of Compound 1;
b. about 120 to 150 mg of microcrystalline cellulose;
c. about 60 to 80 mg anhydrous lactose;
d. about 6 to 18 mg hydroxypropyl cellulose;
e. about 15 to 25 mg of croscarmellose sodium;
f. about 0.4 to 1.5 mg colloidal silicon dioxide; and
g. about 2 to 3 mg magnesium stearate; and optionally
h. about 8 to 26 mg of a film coating.

In one embodiment, the tablet pharmaceutical composition comprises:
a. about 18 to 23 mg of Compound 1;
b. about 30 to 40 mg of microcrystalline cellulose;
c. about 15 to 20 mg anhydrous lactose;
d. about 3 to 7 mg hydroxypropyl cellulose;
e. about 3 to 7 mg of croscarmellose sodium;
f. about 0.1 to 0.3 mg colloidal silicon dioxide; and
g. about 2 to 4 mg stearic acid; and optionally
h. about 2 to 5 mg of a film coating.

In one embodiment, the tablet pharmaceutical composition comprises:
a. about 20 mg of Compound 1;
b. about 30 to 40 mg of microcrystalline cellulose;
c. about 15 to 20 mg anhydrous lactose;
d. about 3 to 7 mg hydroxypropyl cellulose;
e. about 3 to 7 mg of croscarmellose sodium;
f. about 0.1 to 0.3 mg colloidal silicon dioxide; and
g. about 2 to 4 mg stearic acid; and optionally
h. about 2 to 5 mg of a film coating.

In one embodiment, the tablet pharmaceutical composition comprises:
a. about 35 to 45 mg of Compound 1;
b. about 50 to 70 mg of microcrystalline cellulose;
c. about 25 to 35 mg anhydrous lactose;
d. about 6 to 10 mg hydroxypropyl cellulose;
e. about 6 to 10 mg of croscarmellose sodium;
f. about 0.2 to 0.6 mg colloidal silicon dioxide; and
g. about 4 to 8 mg stearic acid; and optionally
h. about 4 to 10 mg of a film coating.

In one embodiment, the tablet pharmaceutical composition comprises:
a. about 40 mg of Compound 1;
b. about 50 to 70 mg of microcrystalline cellulose;
c. about 25 to 35 mg anhydrous lactose;
d. about 6 to 10 mg hydroxypropyl cellulose;
e. about 6 to 10 mg of croscarmellose sodium;
f. about 0.2 to 0.6 mg colloidal silicon dioxide; and
g. about 4 to 8 mg stearic acid; and optionally
h. about 4 to 10 mg of a film coating.

In one embodiment, the tablet pharmaceutical composition comprises:
a. about 55 to 65 mg of Compound 1;
b. about 80 to 120 mg of microcrystalline cellulose;
c. about 40 to 70 mg anhydrous lactose;
d. about 12 to 15 mg hydroxypropyl cellulose;
e. about 12 to 15 mg of croscarmellose sodium;
f. about 0.5 to 0.8 mg colloidal silicon dioxide; and
g. about 6 to 12 mg stearic acid; and optionally
h. about 6 to 12 mg of a film coating.

In one embodiment, the tablet pharmaceutical composition comprises:
a. about 60 mg of Compound 1;
b. about 80 to 120 mg of microcrystalline cellulose;
c. about 40 to 70 mg anhydrous lactose;
d. about 12 to 15 mg hydroxypropyl cellulose;
e. about 12 to 15 mg of croscarmellose sodium;
f. about 0.5 to 0.8 mg colloidal silicon dioxide; and
g. about 6 to 12 mg stearic acid; and optionally
h. about 6 to 12 mg of a film coating.

In one embodiment, the tablet pharmaceutical composition comprises:
a. about 70 to 90 mg of Compound 1;
b. about 120 to 150 mg of microcrystalline cellulose;
c. about 60 to 80 mg anhydrous lactose;
d. about 12 to 30 mg hydroxypropyl cellulose;
e. about 12 to 30 mg of croscarmellose sodium;
f. about 0.5 to 1.5 mg colloidal silicon dioxide; and
g. about 8 to 16 mg stearic acid; and optionally
h. about 8 to 14 mg of a film coating.

In one embodiment, the tablet pharmaceutical composition comprises:
a. about 80 mg of Compound 1;
b. about 120 to 150 mg of microcrystalline cellulose;
c. about 60 to 80 mg anhydrous lactose;
d. about 12 to 30 mg hydroxypropyl cellulose;
e. about 12 to 30 mg of croscarmellose sodium;
f. about 0.5 to 1.5 mg colloidal silicon dioxide; and
g. about 8 to 16 mg stearic acid; and optionally
h. about 8 to 14 mg of a film coating.

In one embodiment, the tablet pharmaceutical composition comprises:
a. about 100 mg of Compound 1;
b. about 140 to 160 mg of microcrystalline cellulose;
c. about 70 to 90 mg anhydrous lactose;
d. about 15 to 25 mg hydroxypropyl cellulose;
e. about 20 to 30 mg of croscarmellose sodium;
f. about 0.8 to 2.0 mg colloidal silicon dioxide; and
g. about 9 to 18 mg stearic acid; and optionally
h. about 10 to 30 mg of a film coating.

In one embodiment, the tablet pharmaceutical composition comprises:
a. about 120 mg of Compound 1;
b. about 165 to 195 mg of microcrystalline cellulose;
c. about 80 to 100 mg anhydrous lactose;
d. about 20 to 30 mg hydroxypropyl cellulose;
e. about 25 to 35 mg of croscarmellose sodium;
f. about 1.0 to 2.5 mg colloidal silicon dioxide; and
g. about 10 to 20 mg stearic acid; and optionally
h. about 15 to 35 mg of a film coating.

### Compound Crystalline Solid Forms

As provided herein, Compound 1 may be present in the pharmaceutical compositions of this disclosure as a crystalline (freebase) solid form or a crystalline salt.

Thus, in one embodiment, the tablet pharmaceutical composition comprises:
a. about 20 percent to about 40 percent by weight of Compound 1 as a crystalline solid or as a crystalline salt;
b. about 35 percent to about 45 percent by weight of microcrystalline cellulose;
c. about 15 to about 25 percent by weight of lactose;
d. about 2 to about 8 percent by weight of hydroxypropyl cellulose;
e. about 4 to about 8 percent by weight of croscarmellose sodium;
f. about 0.1 to about 0.5 percent by weight of silicon dioxide; and
g. about 0.5 to about 3.5 percent by weight magnesium stearate; and optionally
h. a film coating.

In another embodiment, the tablet pharmaceutical composition comprises:
a. about 20 percent to about 40 percent by weight of Compound 1 as a crystalline solid or as a crystalline salt selected from the group consisting of Compound 1 HCl salt, Compound 1 fumaric Salt, and Compound 1 phosphoric acid salt;
b. about 35 percent to about 45 percent by weight of microcrystalline cellulose;
c. about 15 to about 25 percent by weight of lactose;
d. about 2 to about 8 percent by weight of hydroxypropyl cellulose;
e. about 4 to about 8 percent by weight of croscarmellose sodium;
f. about 0.1 to about 0.5 percent by weight of silicon dioxide; and
g. about 0.5 to about 3.5 percent by weight magnesium stearate; and optionally
h. a film coating.

In one embodiment, the tablet pharmaceutical composition comprises:
a. about 20 percent to about 40 percent by weight of Compound 1 as a crystalline solid or as a crystalline salt selected from the group consisting of Compound 1 HCl salt, Compound 1 fumaric Salt, and Compound 1 phosphoric acid salt;
b. about 35 percent to about 45 percent by weight of microcrystalline cellulose;
c. about 15 to about 25 percent by weight of anhydrous lactose;
d. about 2 to about 8 percent by weight of hydroxypropyl cellulose;
e. about 4 to about 8 percent by weight of croscarmellose sodium;
f. about 0.1 to about 0.5 percent by weight of colloidal silicon dioxide; and
g. about 0.5 to about 3.5 percent by weight magnesium stearate; and optionally
h. a film coating.

In one embodiment, the tablet pharmaceutical composition comprises:
a. about 25 percent to about 35 percent by weight of Compound 1 as a crystalline solid or as a crystalline salt selected from the group consisting of Compound 1 HCl salt, Compound 1 fumaric Salt, and Compound 1 phosphoric acid salt;
b. about 37 percent to about 43 percent by weight of microcrystalline cellulose;
c. about 18 to about 22 percent by weight of anhydrous lactose;
d. about 2 to about 6 percent by weight of hydroxypropyl cellulose;
e. about 5 to about 7 percent by weight of croscarmellose sodium;
f. about 0.2 to about 0.4 percent by weight of colloidal silicon dioxide; and
g. about 0.5 to about 3.5 percent by weight magnesium stearate; and optionally
h. a film coating.

In one embodiment, the tablet pharmaceutical composition comprises:
a. about 25 percent to about 35 percent by weight of Compound 1 as a crystalline solid or as a crystalline salt selected from the group consisting of Compound 1 HCl salt, Compound 1 fumaric Salt, and Compound 1 phosphoric acid salt;
b. about 37 percent to about 43 percent by weight of microcrystalline cellulose;
c. about 18 to about 22 percent by weight of anhydrous lactose;
d. about 2 to about 4 percent by weight of hydroxypropyl cellulose;
e. about 5 to about 7 percent by weight of croscarmellose sodium;
f. about 0.2 to about 0.4 percent by weight of colloidal silicon dioxide; and
g. about 0.5 to about 1.5 percent by weight magnesium stearate; and optionally
h. a film coating.

Thus, in another embodiment, the tablet pharmaceutical composition comprises:
a. about 20 percent to about 40 percent by weight of Compound 1 as a crystalline solid or as a crystalline salt selected from the group consisting of Compound 1 HCl salt, Compound 1 fumaric Salt, and Compound 1 phosphoric acid salt;
b. about 35 percent to about 45 percent by weight of microcrystalline cellulose;
c. about 15 to about 25 percent by weight of lactose;
d. about 2 to about 8 percent by weight of hydroxypropyl cellulose;
e. about 2 to about 8 percent by weight of croscarmellose sodium;
f. about 0.1 to about 0.5 percent by weight of silicon dioxide; and
g. about 1 to about 5 percent by weight of stearic acid; and optionally
h. a film coating.

Thus, in another embodiment, the tablet pharmaceutical composition comprises:
a. about 20 percent to about 40 percent by weight of Compound 1 as a crystalline solid or as a crystalline salt selected from the group consisting of Compound 1 HCl salt, Compound 1 fumaric Salt, and Compound 1 phosphoric acid salt;
b. about 35 percent to about 45 percent by weight of microcrystalline cellulose;
c. about 15 to about 25 percent by weight of anhydrous lactose;
d. about 2 to about 8 percent by weight of hydroxypropyl cellulose;
e. about 2 to about 8 percent by weight of croscarmellose sodium;
f. about 0.1 to about 0.5 percent by weight of colloidal silicon dioxide; and
g. about 1 to about 5 percent by weight of stearic acid; and optionally
h. a film coating.

In one embodiment, the tablet pharmaceutical composition comprises:
a. about 25 percent to about 35 percent by weight of Compound 1 as a crystalline solid or as a crystalline salt selected from the group consisting of Compound 1 HCl salt, Compound 1 fumaric Salt, and Compound 1 phosphoric acid salt;
b. about 35 percent to about 40 percent by weight of microcrystalline cellulose;
c. about 16 to about 22 percent by weight of anhydrous lactose;
d. about 3 to about 7 percent by weight of hydroxypropyl cellulose;
e. about 3 to about 7 percent by weight of croscarmellose sodium
f. about 0.1 to about 0.5 percent by weight of colloidal silicon dioxide; and
g. about 0.5 to about 3.5 percent by weight stearic acid; and optionally
h. a film coating.

In one embodiment, the tablet pharmaceutical composition comprises:
a. about 25 percent to about 35 percent by weight of Compound 1 as a crystalline solid or as a crystalline salt selected from the group consisting of Compound 1 HCl salt, Compound 1 fumaric Salt, and Compound 1 phosphoric acid salt;
b. about 35 percent to about 40 percent by weight of microcrystalline cellulose;
c. about 16 to about 22 percent by weight of anhydrous lactose;
d. about 3 to about 7 percent by weight of hydroxypropyl cellulose;
e. about 3 to about 7 percent by weight of croscarmellose sodium
f. about 0.1 to about 0.5 percent by weight of colloidal silicon dioxide; and
g. about 1.5 to about 3.5 percent by weight stearic acid; and optionally
h. a film coating.

In one embodiment, the pharmaceutical compositions of this disclosure comprise Compound 1 as a crystalline (freebase) solid.

In one embodiment, the crystalline solid form of Compound 1 is characterized as Form A, Form B, Form C, Form D, Form E, Form F, Form G, Form H, Form I, Form J, Form K, Form L, Form M, Form N, Form O, Form P, or Form Q. In another embodiment, the crystalline solid form of Compound 1 is characterized as Form A, Form B, Form C, Form D, Form E, Form F, Form G, Form H, Form K, Form O, or Form Q. In another embodiment, the crystalline solid form of Compound 1 is characterized as Form I, Form J, Form L, Form M, Form N, or Form P. The crystalline solid form of Compound 1 characterized as Form A, Form B, Form C, Form D, Form E, Form F, Form G, Form H, Form I, Form J, Form K, Form L, Form M, Form N, Form O, Form P, or Form Q is disclosed in WO 2020/123800, the content of which is incorporated herein by reference in its entirety for all purposes.

In one embodiment, the crystalline solid is characterized as Compound 1 Form A.

In one embodiment, the crystalline solid is characterized as Compound 1 Form B.

In one embodiment, the crystalline solid is characterized as Compound 1 Form C.

In one embodiment, the crystalline solid is characterized as Compound 1 Form D.

In one embodiment, the crystalline solid is characterized as Compound 1 Form E.

In one embodiment, the crystalline solid is characterized as Compound 1 Form F.

In one embodiment, the crystalline solid is characterized as Compound 1 Form G.

In one embodiment, the crystalline solid is characterized as Compound 1 Form H.

In one embodiment, the crystalline solid is characterized as Compound 1 Form I.

In one embodiment, the crystalline solid is characterized as Compound 1 Form J.

In one embodiment, the crystalline solid is characterized as Compound 1 Form K.

In one embodiment, the crystalline solid is characterized as Compound 1 Form L.

In one embodiment, the crystalline solid is characterized as Compound 1 Form M.

In one embodiment, the crystalline solid is characterized as Compound 1 Form N.

In one embodiment, the crystalline solid is characterized as Compound 1 Form 0.

In one embodiment, the crystalline solid is characterized as Compound 1 Form P.

In one embodiment, the crystalline solid is characterized as Compound 1 Form Q.

### Compound 1 Crystalline Salt Forms

In another embodiment, the pharmaceutical compositions of this disclosure comprise Compound 1 as a crystalline salt or a hydrate or solvate thereof.

In one embodiment, the crystalline salt is characterized as Compound 1 HCl Form A, Compound 1 HCl Form B. Compound 1 HCl Form C, or Compound 1 HCl Form D. The crystalline salt form characterized as Compound 1 HCl Form A, Compound 1 HCl Form B, Compound 1 HCl Form C, or Compound 1 HCl Form D is disclosed in WO 2020/123800, the content of which is incorporated herein by reference in its entirety for all purposes.

In one embodiment, the crystalline salt is characterized as Compound 1 HCl Form A.

In one embodiment, the crystalline salt is characterized as Compound 1 HCl Form B.

In one embodiment, the crystalline salt is characterized as Compound 1 HCl Form C.

In one embodiment, the crystalline salt is characterized as Compound 1 HCl Form D.

In one embodiment, the pharmaceutical composition as disclosed herein comprises a crystalline fumaric acid salt of Compound 1, or hydrate or solvate thereof. In some embodiments, the crystalline fumaric acid salt of Compound 1 is characterized as Compound 1 fumarate Form A or Compound 1 hemifumarate Form B. The crystalline fumaric acid salt of Compound 1 characterized as Compound 1 fumarate Form A or Compound 1 hemifumarate Form B is disclosed in in WO 2020/123800, the content of which is incorporated herein by reference in its entirety for all purposes.

In one embodiment, the crystalline salt is characterized as Compound 1 Fumarate Form A.

In one embodiment, the crystalline fumaric acid salt is characterized as Compound 1 hemifumarate Form B.

In one embodiment, the pharmaceutical composition comprises a crystalline phosphoric acid salt of Compound 1 or hydrate or solvate thereof. In some embodiments, the crystalline phosphoric acid salt of Compound 1 is characterized as Compound 1 phosphate Form A. The crystalline phosphoric acid salt of Compound 1 characterized as Compound 1 phosphate Form A is disclosed in in WO 2020/123800, the content of which is incorporated herein by reference in its entirety for all purposes.

In one embodiment, the tablet pharmaceutical composition comprises:
a. about 25 percent to about 35 percent by weight of Compound 1 hemifumarate salt;
b. about 37 percent to about 43 percent by weight of microcrystalline cellulose;
c. about 18 to about 22 percent by weight of anhydrous lactose;
d. about 2 to about 6 percent by weight of hydroxypropyl cellulose;
e. about 5 to about 7 percent by weight of croscarmellose sodium;
f. about 0.2 to about 0.4 percent by weight of colloidal silicon dioxide; and
g. about 0.5 to about 3.5 percent by weight magnesium stearate; and optionally
h. a film coating.

In one embodiment, the tablet pharmaceutical composition comprises:
a. about 25 percent to about 35 percent by weight of Compound 1 hemifumarate salt;
b. about 37 percent to about 43 percent by weight of microcrystalline cellulose;
c. about 18 to about 22 percent by weight of anhydrous lactose;
d. about 2 to about 4 percent by weight of hydroxypropyl cellulose;
e. about 5 to about 7 percent by weight of croscarmellose sodium;
f. about 0.2 to about 0.4 percent by weight of colloidal silicon dioxide; and
g. about 0.5 to about 1.5 percent by weight magnesium stearate; and optionally
h. a film coating.

In one embodiment, the tablet pharmaceutical composition comprises:
a. about 25 percent to about 35 percent by weight of Compound 1 hemifumarate salt Form B;
b. about 37 percent to about 43 percent by weight of microcrystalline cellulose;
c. about 18 to about 22 percent by weight of anhydrous lactose;
d. about 2 to about 6 percent by weight of hydroxypropyl cellulose;
e. about 5 to about 7 percent by weight of croscarmellose sodium;
f. about 0.2 to about 0.4 percent by weight of colloidal silicon dioxide; and
g. about 0.5 to about 3.5 percent by weight magnesium stearate; and optionally
h. a film coating.

In one embodiment, the tablet pharmaceutical composition comprises:
a. about 27.75 percent by weight of Compound 1 hemifumarate salt;
b. about 41.47 percent by weight of microcrystalline cellulose;
c. about 20.73 percent by weight of anhydrous lactose;
d. about 3 percent by weight of hydroxypropyl cellulose;
e. about 6 percent by weight of croscarmellose sodium;
f. about 0.3 percent by weight of colloidal silicon dioxide; and
g. about 0.75 percent by weight magnesium stearate; and optionally
h. a film coating.

In one embodiment, the tablet pharmaceutical composition comprises:
a. about 27.75 percent by weight of Compound 1 hemifumarate salt Form B;
b. about 41.47 percent by weight of microcrystalline cellulose;
c. about 20.73 percent by weight of anhydrous lactose;
d. about 3 percent by weight of hydroxypropyl cellulose;
e. about 6 percent by weight of croscarmellose sodium;
f. about 0.3 percent by weight of colloidal silicon dioxide; and
g. about 0.75 percent by weight magnesium stearate; and optionally
h. a film coating.

In one embodiment, the tablet pharmaceutical composition comprises:
a. about 20 to 25 mg of Compound 1 hemifumarate salt;
b. about 30 to 35 mg of microcrystalline cellulose;
c. about 15 to 18 mg anhydrous lactose;
d. about 1.5 to 4.5 mg hydroxypropyl cellulose;
e. about 4 to 6 mg of croscarmellose sodium;
f. about 0.1 to 0.3 mg colloidal silicon dioxide; and
g. about 0.5 to 0.7 mg magnesium stearate; and optionally
h. about 2 to 6 mg of a film coating.

In one embodiment, the tablet pharmaceutical composition comprises:
a. about 20 to 25 mg of Compound 1 hemifumarate salt Form B;
b. about 30 to 35 mg of microcrystalline cellulose;
c. about 15 to 18 mg anhydrous lactose;
d. about 1.5 to 4.5 mg hydroxypropyl cellulose;
e. about 4 to 6 mg of croscarmellose sodium;
f. about 0.1 to 0.3 mg colloidal silicon dioxide; and
g. about 0.5 to 0.7 mg magnesium stearate; and optionally
h. about 2 to 6 mg of a film coating.

In one embodiment, the tablet pharmaceutical composition comprises:
a. about 22.20 mg of Compound 1 hemifumarate salt Form B;
b. about 30 to 35 mg of microcrystalline cellulose;
c. about 15 to 18 mg anhydrous lactose;
d. about 1.5 to 4.5 mg hydroxypropyl cellulose;
e. about 4 to 6 mg of croscarmellose sodium;
f. about 0.1 to 0.3 mg colloidal silicon dioxide; and
g. about 0.5 to 0.7 mg magnesium stearate; and optionally
h. about 2 to 6 mg of a film coating.

In one embodiment, the tablet pharmaceutical composition comprises:
a. about 22.20 mg of Compound 1 hemifumarate salt Form B;
b. about 33.17 mg of microcrystalline cellulose;
c. about 16.59 mg anhydrous lactose;
d. about 2.4 mg hydroxypropyl cellulose;
e. about 4.8 mg of croscarmellose sodium;
f. about 0.24 mg colloidal silicon dioxide; and
g. about 0.6 mg magnesium stearate; and optionally
h. about 3.2 mg of a film coating.

In one embodiment, the tablet pharmaceutical composition comprises:
a. about 25 percent to about 35 percent by weight of Compound 1 hemifumarate salt;
b. about 35 percent to about 40 percent by weight of microcrystalline cellulose;
c. about 16 to about 22 percent by weight of anhydrous lactose;
d. about 3 to about 7 percent by weight of hydroxypropyl cellulose;
e. about 3 to about 7 percent by weight of croscarmellose sodium
f. about 0.1 to about 0.5 percent by weight of colloidal silicon dioxide; and
g. about 0.5 to about 3.5 percent by weight stearic acid; and optionally
h. a film coating.

In one embodiment, the tablet pharmaceutical composition comprises:
a. about 25 percent to about 35 percent by weight of Compound 1 hemifumarate salt;
b. about 35 percent to about 40 percent by weight of microcrystalline cellulose;
c. about 16 to about 22 percent by weight of anhydrous lactose;
d. about 3 to about 7 percent by weight of hydroxypropyl cellulose;
e. about 3 to about 7 percent by weight of croscarmellose sodium
f. about 0.1 to about 0.5 percent by weight of colloidal silicon dioxide; and
g. about 1.5 to about 3.5 percent by weight stearic acid; and optionally
h. a film coating.

In one embodiment, the tablet pharmaceutical composition comprises:
a. about 25 percent to about 35 percent by weight of Compound 1 hemifumarate salt Form B;
b. about 35 percent to about 40 percent by weight of microcrystalline cellulose;
c. about 16 to about 22 percent by weight of anhydrous lactose;
d. about 3 to about 7 percent by weight of hydroxypropyl cellulose;
e. about 3 to about 7 percent by weight of croscarmellose sodium
f. about 0.1 to about 0.5 percent by weight of colloidal silicon dioxide; and
g. about 0.5 to about 3.5 percent by weight stearic acid; and optionally
h. a film coating.

In one embodiment, the tablet pharmaceutical composition comprises:
a. about 27.75 percent by weight of Compound 1 hemifumarate salt;
b. about 38.63 percent by weight of microcrystalline cellulose;
c. about 19.32 percent by weight of anhydrous lactose;
d. about 5 percent by weight of hydroxypropyl cellulose;
e. about 6 percent by weight of croscarmellose sodium
f. about 0.3 percent by weight of colloidal silicon dioxide; and
g. about 3 percent by weight stearic acid; and optionally
h. a film coating.

In one embodiment, the tablet pharmaceutical composition comprises:
a. about 27.75 percent by weight of Compound 1 hemifumarate salt Form B;
b. about 38.63 percent by weight of microcrystalline cellulose;
c. about 19.32 percent by weight of anhydrous lactose;
d. about 5 percent by weight of hydroxypropyl cellulose;
e. about 6 percent by weight of croscarmellose sodium
f. about 0.3 percent by weight of colloidal silicon dioxide; and
g. about 3 percent by weight stearic acid; and optionally
h. a film coating.

In one embodiment, the tablet pharmaceutical composition comprises:
a. about 20 to 25 mg of Compound 1 hemifumarate salt;
b. about 30 to 40 mg of microcrystalline cellulose;
c. about 15 to 20 mg anhydrous lactose;
d. about 3 to 7 mg hydroxypropyl cellulose;
e. about 3 to 7 mg of croscarmellose sodium;
f. about 0.1 to 0.3 mg colloidal silicon dioxide; and
g. about 2 to 4 mg stearic acid; and optionally
h. about 2 to 5 mg of a film coating.

In one embodiment, the tablet pharmaceutical composition comprises:
a. about 20 to 25 mg of Compound 1 hemifumarate salt Form B;
b. about 30 to 40 mg of microcrystalline cellulose;
c. about 15 to 20 mg anhydrous lactose;
d. about 3 to 7 mg hydroxypropyl cellulose;
e. about 3 to 7 mg of croscarmellose sodium;
f. about 0.1 to 0.3 mg colloidal silicon dioxide; and
g. about 2 to 4 mg stearic acid; and optionally
h. about 2 to 5 mg of a film coating.

In one embodiment, the tablet pharmaceutical composition comprises:
a. about 22.20 mg of Compound 1 hemifumarate salt Form B;
b. about 30 to 40 mg of microcrystalline cellulose;
c. about 15 to 20 mg anhydrous lactose;
d. about 3 to 7 mg hydroxypropyl cellulose;
e. about 3 to 7 mg of croscarmellose sodium;
f. about 0.1 to 0.3 mg colloidal silicon dioxide; and
g. about 2 to 4 mg stearic acid; and optionally
h. about 2 to 5 mg of a film coating.

In one embodiment, the tablet pharmaceutical composition comprises:
a. about 22.20 mg of Compound 1 hemifumarate salt Form B;
b. about 30.9 mg of microcrystalline cellulose;
c. about 15.46 mg anhydrous lactose;
d. about 4 mg hydroxypropyl cellulose;
e. about 4.8 mg of croscarmellose sodium;
f. about 0.24 mg colloidal silicon dioxide; and
g. about 2.4 mg stearic acid; and optionally
h. about 3.2 mg of a film coating.

In one embodiment, the tablet pharmaceutical composition comprises:
a. about 83 to 93 mg of Compound 1 hemifumarate salt;
b. about 120 to 150 mg of microcrystalline cellulose;
c. about 60 to 80 mg anhydrous lactose;
d. about 12 to 30 mg hydroxypropyl cellulose;
e. about 12 to 30 mg of croscarmellose sodium;
f. about 0.5 to 1.5 mg colloidal silicon dioxide; and
g. about 8 to 16 mg stearic acid; and optionally
h. about 8 to 14 mg of a film coating.

In one embodiment, the tablet pharmaceutical composition comprises:
a. about 83 to 93 mg of Compound 1 hemifumarate salt Form B;
b. about 120 to 150 mg of microcrystalline cellulose;
c. about 60 to 80 mg anhydrous lactose;
d. about 12 to 30 mg hydroxypropyl cellulose;
e. about 12 to 30 mg of croscarmellose sodium;
f. about 0.5 to 1.5 mg colloidal silicon dioxide; and
g. about 8 to 16 mg stearic acid; and optionally
h. about 8 to 14 mg of a film coating.

In one embodiment, the tablet pharmaceutical composition comprises:
a. about 88.78 mg of Compound 1 hemifumarate salt Form B;
b. about 120 to 150 mg of microcrystalline cellulose;
c. about 60 to 80 mg anhydrous lactose;
d. about 12 to 30 mg hydroxypropyl cellulose;
e. about 12 to 30 mg of croscarmellose sodium;
f. about 0.5 to 1.5 mg colloidal silicon dioxide; and
g. about 8 to 16 mg stearic acid; and optionally
h. about 8 to 14 mg of a film coating.

In one embodiment, the tablet pharmaceutical composition comprises:
a. about 88.78 mg of Compound 1 hemifumarate salt Form B;
b. about 123.62 mg of microcrystalline cellulose;
c. about 61.82 mg anhydrous lactose;
d. about 16 mg hydroxypropyl cellulose;
e. about 19.2 mg of croscarmellose sodium;
f. about 0.96 mg colloidal silicon dioxide; and
g. about 9.6 mg stearic acid; and optionally
h. about 12.8 mg of a film coating.

In one embodiment, the tablet pharmaceutical composition comprises:
a. about 100 to 120 mg of Compound 1 hemifumarate salt Form B;
b. about 140 to 160 mg of microcrystalline cellulose;
c. about 70 to 90 mg anhydrous lactose;
d. about 15 to 25 mg hydroxypropyl cellulose;
e. about 20 to 30 mg of croscarmellose sodium;
f. about 0.8 to 2.0 mg colloidal silicon dioxide; and
g. about 9 to 18 mg stearic acid; and optionally
h. about 10 to 30 mg of a film coating.

In one embodiment, the tablet pharmaceutical composition comprises:
a. about 111 mg of Compound 1 hemifumarate salt Form B;
b. about 140 to 160 mg of microcrystalline cellulose;
c. about 70 to 90 mg anhydrous lactose;
d. about 15 to 25 mg hydroxypropyl cellulose;
e. about 20 to 30 mg of croscarmellose sodium;
f. about 0.8 to 2.0 mg colloidal silicon dioxide; and
g. about 9 to 18 mg stearic acid; and optionally
h. about 10 to 30 mg of a film coating.

In one embodiment, the tablet pharmaceutical composition comprises:
a. about 111 mg of Compound 1 hemifumarate salt Form B;
b. about 154.52 mg of microcrystalline cellulose;
c. about 77.28 mg anhydrous lactose;
d. about 20 mg hydroxypropyl cellulose;
e. about 24 mg of croscarmellose sodium;
f. about 1.2 mg colloidal silicon dioxide; and
g. about 12 mg stearic acid; and optionally
h. about 16 mg of a film coating.

In one embodiment, the tablet pharmaceutical composition comprises:
a. about 130 to 140 mg of Compound 1 hemifumarate salt Form B;
b. about 165 to 195 mg of microcrystalline cellulose;
c. about 80 to 100 mg anhydrous lactose;
d. about 20 to 30 mg hydroxypropyl cellulose;
e. about 25 to 35 mg of croscarmellose sodium;
f. about 1.0 to 2.5 mg colloidal silicon dioxide; and
g. about 10 to 20 mg stearic acid; and optionally
h. about 15 to 35 mg of a film coating.

In one embodiment, the tablet pharmaceutical composition comprises:
a. about 132 mg of Compound 1 hemifumarate salt Form B;
b. about 165 to 195 mg of microcrystalline cellulose;
c. about 80 to 100 mg anhydrous lactose;
d. about 20 to 30 mg hydroxypropyl cellulose;
e. about 25 to 35 mg of croscarmellose sodium;
f. about 1.0 to 2.5 mg colloidal silicon dioxide; and
g. about 10 to 20 mg stearic acid; and optionally
h. about 15 to 35 mg of a film coating.

In one embodiment, the tablet pharmaceutical composition comprises:
a. about 132 mg of Compound 1 hemifumarate salt Form B;
b. about 185.42 mg of microcrystalline cellulose;
c. about 92.74 mg anhydrous lactose;
d. about 24 mg hydroxypropyl cellulose;
e. about 28.8 mg of croscarmellose sodium;
f. about 1.44 mg colloidal silicon dioxide; and
g. about 14.4 mg stearic acid; and optionally
h. about 19.2 mg of a film coating.

### Methods of Treatment

In still another aspect, the invention relates to a method of treating a disease, disorder, or syndrome mediated at least in part by modulating in vivo activity of a protein kinase, comprising administering to a subject in need thereof a pharmaceutical composition of a crystalline form or a crystalline salt form of Compound 1 as described herein.

In one embodiment of this aspect, the disease, disorder, or syndrome mediated at least in part by modulating in vivo activity of a protein kinase is cancer.

In one embodiment, the cancer is selected from cardiac cancer, head and neck cancer, lung cancer, colon cancer, gastrointestinal cancer, breast cancer, genitourinary tract cancer, liver cancer, bone cancer, thyroid cancer, cancer of the nervous system, gynecological cancer, hematologic cancer, skin cancer, and cancer of the adrenal glands.

In a further embodiment, the cardiac cancer is selected from angiosarcoma, fibrosarcoma, rhabdomyosarcoma, liposarcoma, myxoma, rhabdomyoma, fibroma, lipoma, and teratoma.

In another further embodiment, the head and neck cancer is selected from squamous cell carcinomas of the head and neck, laryngeal and hypopharyngeal cancer, nasal cavity and paranasal sinus cancer, nasopharyngeal cancer, salivary gland cancer, oral and oropharyngeal cancer.

In another further embodiment, the lung cancer is selected from bronchogenic carcinomas selected from squamous cell, undifferentiated small cell, undifferentiated large cell, adenocarcinoma, and non-small cell lung cancer; alveolar (bronchiolar) carcinoma bronchial adenoma sarcoma lymphoma chondromatous hamartoma and mesothelioma.

In another further embodiment, the colon cancer is selected from colorectal cancer, adenocarcinoma, gastrointestinal stromal tumors, lymphoma, carcinoids, and Turcot Syndrome.

In another further embodiment, the gastrointestinal cancer is selected from gastric cancer, gastroesophageal junction adenocarcinoma, esophageal squamous cell carcinoma, esophageal adenocarcinoma, esophageal leiomyosarcoma, esophageal lymphoma, gastric carcinoma, gastric lymphoma, gastric leiomyosarcoma, pancreatic ductal adenocarcinoma, pancreatic insulinoma, pancreatic glucagonoma, pancreatic gastrinoma, pancreatic carcinoid tumors, vipoma, small intestinal adenocarcinoma, small intestinal lymphoma, small intestinal carcinoid tumors, small intestinal Karposi's sarcoma, small intestinal leiomyoma, small intestinal hemangioma, small intestinal lipoma, small intestinal neurofibroma, small intestinal fibroma, large intestinal adenocarcinoma, large intestinal tubular adenoma, large intestinal villous adenoma, large intestinal hamartoma, and large intestinal leiomyoma.

In another further embodiment, the breast cancer is selected from metastatic breast cancer, ductal carcinoma in situ, invasive ductal carcinoma, tubular carcinoma, medullary carcinoma, mucinous carcinoma, lobular carcinoma in situ, and triple negative breast cancer;

In another further embodiment, the genitourinary tract cancer is selected from renal adenocarcinoma, renal nephroblastoma, renal lymphoma, renal cell carcinoma, squamous cell carcinoma of the bladder or urethra, transitional cell carcinoma of the bladder or urethra, adenocarcinoma of the bladder or urethra, urothelial carcinoma of the bladder or urethra, prostate adenocarcinoma, prostate sarcoma, castrate resistant prostate cancer, seminoma, testicular teratoma, embryonal carcinoma, testicular teratocarcinoma, testicular choriocarcinoma, testicular sarcoma, testicular interstitial cell carcinoma, testicular fibroma, testicular fibroadenoma, testicular adenomatoid tumors, testicular lipoma, clear cell carcinoma, and papillary carcinoma.

In another further embodiment, the liver cancer is selected from hepatocellular carcinoma, cholangiocarcinoma, hepatoblastoma, angiosarcoma, hepatocellular adenoma, and hemangioma.

In another further embodiment, the bone cancer is selected from osteogenic sarcoma, fibrosarcoma, malignant fibrous histiocytoma, chondrosarcoma, Ewing's sarcoma, malignant lymphoma, reticulum cell sarcoma, multiple myeloma, malignant giant cell tumor chordoma, osteochrondroma, benign chondroma, chondroblastoma, chondromyxofibroma, osteoid osteoma, and giant cell tumors.

In another further embodiment, the thyroid cancer is selected from medullary thyroid cancer, differentiated thyroid cancer, papillary thyroid cancer, follicular thyroid cancer, hurthle cell cancer, and anaplastic thyroid cancer;

In another further embodiment, the nervous system cancer is selected from osteoma of the skull, hemangioma of the skull, granuloma of the skull, xanthoma of the skull, osteitis deformans of the skull, meningioma, meningiosarcoma, gliomatosis of the meninges, brain astrocytoma, medulloblastoma, glioma, brain ependymoma, germinoma [pinealoma], glioblastoma multiform, oligodendroglioma, schwannoma, retinoblastoma, congenital brain tumors, spinal cord neurofibroma, meningioma, and brain sarcoma.

In another further embodiment, the gynecological cancer is selected from endometrial cancer, cervical carcinoma, pre-tumor cervical dysplasia, ovarian carcinomas selected from serous cystadenocarcinoma, mucinous cystadenocarcinoma, and unclassified ovarian carcinoma, granulosa-thecal cell tumors, Sertoli-Leydig cell tumors, dysgerminoma, and malignant teratoma; squamous cell carcinoma of the vulva, intraepithelial carcinoma of the vulva, adenocarcinoma of the vulva, fibrosarcoma of the vulva, melanoma of the vulva, vaginal clear cell carcinoma, vaginal squamous cell carcinoma, embryonal rhabdomyosarcoma, and fallopian tube carcinoma.

In another further embodiment, the hematologic cancer is selected from myeloid leukemia [acute and chronic], acute lymphoblastic leukemia, chronic lymphocytic leukemia, myeloproliferative diseases, multiple myeloma, myelodysplastic syndrome), Hodgkin's disease, and non-Hodgkin's lymphoma [malignant lymphoma].

In another further embodiment, the skin cancer is selected from malignant melanoma, basal cell carcinoma, squamous cell carcinoma, Karposi's sarcoma, moles dysplastic nevi, lipoma, angioma, dermatofibroma, keloids, and psoriasis.

In another further embodiment, the adrenal gland cancer is neuroblastoma.

In another further embodiment, the cancer is advanced clear cell renal cell carcinoma, hormone receptor positive breast cancer, or castration resistant prostate cancer.

In another further embodiment, the cancer is advanced clear cell renal cell carcinoma.

In another further embodiment, the cancer is hormone receptor positive breast cancer.

In another further embodiment, the cancer is castration resistant prostate cancer.

In another embodiment, the cancer is non-clear cell renal cell carcinoma.

In another embodiment, the cancer is clear cell renal cell carcinoma.

### Labeled Compounds and Assay Methods

Another aspect relates to labeled crystalline forms or crystalline salt forms of the present invention (radio-labeled, fluorescent-labeled, etc.) that would be useful not only in imaging techniques but also in assays, both in vitro and in vivo, for localizing and quantitating TAM kinases in tissue samples, including human, and for identifying TAM kinase ligands by inhibition binding of a labeled compound. Accordingly, the present invention includes TAM kinase assays that contain such labeled compounds.

The present invention further includes isotopically-labeled crystalline forms or crystalline salt forms of the present invention. An “isotopically” or “radio-labeled” compound is a crystalline form or crystalline salt form of the present invention where one or more atoms are replaced or substituted by an atom having an atomic mass or mass number different from the atomic mass or mass number typically found in nature (i.e., naturally occurring). Suitable radionuclides that may be incorporated in crystalline forms or crystalline salt forms of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³, ¹²⁴I, ¹²⁵I and ¹³¹I. The radionuclide that is incorporated in the instant radio-labeled compounds will depend on the specific application of that radio-labeled compound. For example, for in vitro metalloprotease labeling and competition assays, compounds that incorporate ³H, ¹⁴C, ⁸²Br, ¹²⁵I, ¹³¹I, or ³⁵S will generally be most useful. For radio-imaging applications ¹¹C, ¹⁸F, ¹²⁵I, ¹²³I, ¹²⁴I, ¹³¹I, ⁷⁵Br, ⁷⁶Br, or ⁷⁷Br will generally be most useful. In some embodiments, the crystalline forms or crystalline salt forms described herein in which one or more hydrogens is/are replaced by deuterium, such as hydrogen bonded to a carbon atom. Such compounds exhibit increased resistance to metabolism and are thus useful for increasing the half-life of any compound when administered to a mammal, particularly a human.

It is understood that a “radio-labeled” or “labeled compound” is a compound that has incorporated at least one radionuclide. In some embodiments, the radionuclide is selected from the group consisting of ³H, ¹⁴C, ¹²⁵I, ³⁵S, and ⁸²Br.

The present invention can further include synthetic methods for incorporating radio-isotopes into crystalline forms or crystalline salt forms of the present invention. Synthetic methods for incorporating radio-isotopes into organic compounds are well known in the art, and a person of ordinary skill in the art will readily recognize the methods applicable for the compounds of invention.

A labeled compound of the invention can be used in a screening assay to identify/evaluate compounds. For example, a newly synthesized or identified compound (i.e., test compound) which is labeled can be evaluated for its ability to bind a TAM by monitoring its concentration variation when contacting with the TAM kinases, through tracking of the labeling. For example, a test compound (labeled) can be evaluated for its ability to reduce binding of another compound which is known to bind to a TAM kinase (i.e., standard compound). Accordingly, the ability of a test compound to compete with the standard compound for binding to the TAM kinase directly correlates to its binding affinity. Conversely, in some other screening assays, the standard compound is labeled, and test compounds are unlabeled. Accordingly, the concentration of the labeled standard compound is monitored in order to evaluate the competition between the standard compound and the test compound, and the relative binding affinity of the test compound is thus ascertained.

### EXAMPLES

### General Experimental Techniques

Aqueous Slurry Experiments: Salts of Compound 1 that were determined to have aqueous solubility less than 1 mg/mL were slurried in 20 mL of water at ambient temperature for 1 day. Solids were then collected by vacuum filtration and analyzed by XRPD.

Crash Cooling (CC): Concentrated solutions of Compound 1 and various counterions were prepared in MeOH at elevated temperature with stirring. Capped vials containing hot solutions were transferred to the freezer (˜−20° C.) and rapidly cooled. Solids that were formed were collected. If no solids were present, additional crystallization techniques were employed.

Crash Precipitation (CP): Clear solutions of Compound 1 and coformer were prepared in various solvents at RT. Aliquots of various anti-solvents were added to the solution, slowly, with gentle stirring until solids crashed out of solution. Mixtures were allowed to stir for a specified period of time. Solids that formed were collected by positive-pressure filtration.

Fast Cooling (FC): Concentrated solutions of Compound 1 and various counterions were prepared in acetone or MeOH at elevated temperature with stirring. Capped vials containing hot solutions were transferred to the bench top at ambient temperature. Solids that were formed were collected. If no solids were present, additional crystallization techniques were employed.

Fast Evaporation (FE): Clear solutions of Compound 1 and coformer were prepared in various solvents. Vials were left uncapped and solvent evaporated at ambient conditions.

Interconversion Slurry: A slurry of Compound 1 Form A was prepared by adding enough solids to a given solvent system at ambient conditions so that undissolved solids were present. The mixture was then agitated for an extended period of time to ensure saturation. Solids of the forms of interest were then added to an aliquot of the saturated solution (filtered through a 0.2-μm nylon filter) so that undissolved solids were present. The mixture was then agitated at ambient temperature for an extended period of time, and the solids were isolated.

Isolation Techniques: In general, isolation was done quickly after removing non-ambient samples from their respective temperature control devices to minimize equilibration to ambient temperature prior to isolation of the solids.

Decanting Liquid Phase: Some of the solids isolated from solution-based crystallization techniques were collected by centrifuging the suspension (if needed) and discarding the liquid phase, leaving behind damp solids. Solids were dried briefly (e.g., air dried or dried under nitrogen) unless specified as “analyzed damp” herein.

Positive-Pressure Filtration: Solids were collected on 0.2-μm nylon or PTFE filters by pressing a slurry through a syringe and Swinnex filter holder assembly. In general, solids were dried briefly by blowing a 20-mL syringe of air over the filter. If designated as “analyzed damp” herein, solids were left damp with mother liquor. Some samples were additionally dried briefly under a gentle stream of nitrogen gas prior to analysis.

Vacuum Filtration: Solids were collected on paper or nylon filters by vacuum filtration and air dried on the filters under reduced pressure briefly before transferring to a vial.

Reaction Crystallization (RC): A mixture of Compound 1 and various coformers were combined in an elevated temperature, acetone slurry, such that the molarity of coformer was 2-fold greater than the API. The solution stirred for a given period of time. Additional crystallization techniques were employed when clear solutions were observed.

Stability Testing: Various Compound 1 salts were placed in open vials within a stability test chamber (for example, 60% or 75% relative humidity (RH) with saturated sodium chloride solution). The RH chamber was placed in an oven (for example, 25° C. or 40° C.) for a period of time. Samples were analyzed by PLM and XRPD upon the end of the duration.

Dissolution Testing: Dissolution release of Compound 1 in various pharmaceutical compositions of Compound 1 was determined by High performance Liquid Chromatography (HPCL). The pharmaceutical composition or tablet of Compound 1 was placed into a dissolution medium of 0.375% Triton X-100 in 0.01N HCl at a temperature of 37.0±0.5° C. Sample solutions were withdrawn at 5, 10, 20, 30, 45, 60, 90, 120, 10, 180, and 210 minutes time points for the HPLC analysis.

Slow Cooling (SC): Concentrated solutions of Compound 1 and various coformers were prepared in a variety of solvents at elevated temperatures with stirring. Vials were capped in the heated sample block and the hot plate was turned off, allowing the vials to gradually cool to ambient temperature in the heated vial block. Clear solutions, upon cooling to ambient, were further cooled in the refrigerator (5 to 7° C.) and/or the freezer (˜-20° C.). If no solids were present, additional crystallization techniques were employed.

Slow Evaporation: Solutions were prepared in various solvents with agitation and, typically, filtered through a 0.2-μm nylon or PTFE filter. Each solution was allowed to evaporate from a covered vial (such as loosely capped or covered with perforated aluminum foil) at ambient conditions, unless otherwise stated. Solutions were allowed to evaporate to dryness unless designated as partial evaporations (solid present with a small amount of solvent remaining), in which case solids were isolated as described herein.

Solubility Estimation: Aliquots of various solvents were added to measured amounts of Compound 1 with agitation (typically sonication) at stated temperatures until complete dissolution was achieved, as judged by visual observation. If dissolution occurred after the addition of the first aliquot, values are reported as “>.” If dissolution did not occur, values are reported as “<.”

Aqueous Solubility Estimation: Aliquots of water were added to measured amounts of various Compound 1 salts with sonication.

Slurry Experiments: Saturated solutions of Compound 1 and various coformers were prepared in a variety of solvents and solvent mixtures. Mixtures were stirred at ambient and elevated temperatures for the noted duration of time. Solids were collected by the stated technique and additional crystallization techniques were employed where appropriate.

Vacuum Oven Desolvation: Salts of Compound 1 that were determined to be solvates by various analytical methods underwent an attempted desolvation. Samples were placed in a vacuum oven at temperatures ranging from ambient to 80° C. for a given period of time. Samples were analyzed by XRPD and/or TGA for determination of desolvation success.

Vapor Diffusion: Concentrated solutions were prepared in various solvents and, typically, filtered through a 0.2-μm nylon or PTFE filter. The filtered solution was dispensed into a small vial, which was then placed inside a larger vial containing anti-solvent. The small vial was left uncapped and the larger vial was capped to allow vapor diffusion to occur. Any solids present were isolated as described herein.

Vapor Stressing: Select solids were transferred to a small vial, which was then placed inside a larger vial containing solvent. The small vial was left uncapped and the larger vial was capped to allow vapor stressing to occur at the stated temperature.

Coformer means one or more pharmaceutically acceptable bases and/or pharmaceutically acceptable acids disclosed herein in association with Compound 1. Exemplary coformers as used herein include fumaric acid, HCl, and phosphoric acid.

Instrumental Techniques

Differential Scanning Calorimetry (DSC): DSC was performed using a Mettler-Toledo DSC3+ differential scanning calorimeter. Temperature calibration was performed using adamantane, phenyl salicylate, indium, tin, and zinc. The sample was placed into a hermetically sealed or an open aluminum DSC pan, and the weight was accurately recorded. A weighed aluminum pan configured as the sample pan was placed on the reference side of the cell. The samples were analyzed from −30 to 250° C. at a ramp rate of 10° C./min. Although thermograms are plotted by reference temperature (x-axis), results are reported according to sample temperatures.

Dynamic Vapor Sorption (DVS)

a. VTI: Automated vapor sorption (VS) data were collected on a VTI SGA-100 Vapor Sorption Analyzer. NaCl and PVP were used as calibration standards. Samples were dried prior to analysis. Sorption and desorption data were collected over a range from 5% to 95% RH at 10% RH increments under a nitrogen purge. The equilibrium criterion used for analysis was less than 0.0100% weight change in 5 minutes with a maximum equilibration time of 3 hours. Data were not corrected for the initial moisture content of the samples.

b. Intrinsic: Automated vapor sorption (VS) data were collected on a Surface Measurement System DVS Intrinsic instrument. Samples were not dried prior to analysis. Sorption and desorption data were collected over a range from 5% to 95% RH at 10% RH increments under a nitrogen purge. The equilibrium criterion used for analysis was less than 0.0100% weight change in 5 minutes with a maximum equilibration time of 3 hours. Data were not corrected for the initial moisture content of the samples.

Hot stage Microscopy (HSM): Hot stage microscopy was performed using a Linkam hot stage (FTIR 600) mounted on a Leica DM LP microscope equipped with a SPOT Insight™ color digital camera. Temperature calibrations were performed using USP melting point standards. Samples were placed on a cover glass, and a second cover glass was placed on top of the sample. As the stage was heated, each sample was visually observed using a 20× objective with crossed polarizers and a first order red compensator. Images were captured using SPOT software (v. 4.5.9).

Optical Microscopy: Samples were observed under a Motic or Wolfe optical microscope with crossed polarizers or under a Leica stereomicroscope with a first order red compensator with crossed polarizers.

pKa and log P Determination: pKa and log P determination were performed by Pion Inc./Sirius Analytical Instruments Ltd. in East Sussex, United Kingdom.

Solution Proton Nuclear Magnetic Resonance Spectroscopy (¹HNMR): The solution ¹H NMR spectra were acquired by Spectral Data Services of Champaign, IL. The samples were prepared by dissolving approximately 5-10 mg of sample in DMSO-d₆. The data acquisition parameters are displayed on the first page of each spectrum in the Data section of this report.

Thermogravimetric Analysis (TGA): Thermogravimetric analyses were performed using a Mettler Toledo TGA/DSC3+ analyzer. Temperature calibration was performed using phenyl salicylate, indium, tin, and zinc. The sample was placed in an aluminum pan. The open pan was inserted into the TG furnace. The furnace was heated under nitrogen. Each sample was heated from ambient temperature to 350° C., at ramp rates of 2, 5, or 10° C./min. Although thermograms are plotted by reference temperature (x-axis), results are reported according to sample temperatures.

X-ray Powder Diffraction (XRPD)

a. Reflection: XRPD patterns were collected with a PANalytical X'Pert PRO MPD diffractometer using an incident beam of Cu Kα radiation produced using a long, fine-focus source and a nickel filter at room temperature (298 Kelvin). The diffractometer was configured using the symmetric Bragg-Brentano geometry. Prior to the analysis, a silicon specimen (NIST SRM 640e) was analyzed to verify the observed position of the Si 111 peak is consistent with the NIST-certified position. A specimen of the sample was packed in a well. Antiscatter slits (SS) were used to minimize the background generated by air. Soller slits for the incident and diffracted beams were used to minimize broadening from axial divergence. Diffraction patterns were collected using a scanning position-sensitive detector (X'Celerator) located 240 mm from the sample and Data Collector software v. 2.2b. The data acquisition parameters for each pattern are displayed above the image in the Data section of this report including the divergence slit (DS) and the incident-beam SS.

b. Transmission: XRPD patterns were collected with a PANalytical X'Pert PRO MPD diffractometer using an incident beam of Cu radiation produced using an Optix long, fine-focus source at room temperature (298 Kelvin). An elliptically graded multilayer mirror was used to focus Cu Kα X-rays through the specimen and onto the detector. Prior to the analysis, a silicon specimen (NIST SRM 640e) was analyzed to verify the observed position of the Si 111 peak is consistent with the NIST-certified position. A specimen of the sample was sandwiched between 3-μm-thick films and analyzed in transmission geometry. A beam-stop, short antiscatter extension, antiscatter knife edge, were used to minimize the background generated by air. Soller slits for the incident and diffracted beams were used to minimize broadening from axial divergence. Diffraction patterns were collected using a scanning position-sensitive detector (X'Celerator) located 240 mm from the specimen and Data Collector software v. 2.2b. The data acquisition parameters for each pattern are displayed above the image in the Data section of this report including the divergence slit (DS) before the mirror.

XRPD Indexing

Indexing and structure refinement are computational studies. Within the figure referenced for a given indexed XRPD pattern, agreement between the allowed peak positions, marked with bars, and the observed peaks indicates a consistent unit cell determination. Successful indexing of a pattern indicates that the sample is composed primarily of a single crystalline phase unless otherwise stated. Space groups consistent with the assigned extinction symbol, unit cell parameters, and derived quantities are tabulated.

### Compound Preparation Examples

### Preparative Example 1: Synthesis of Compound 1

### Step 1: N-(4-Fluorophenyl)-N-(4-hydroxyphenyl)cyclopropane-1,1-dicarboxamide (4)

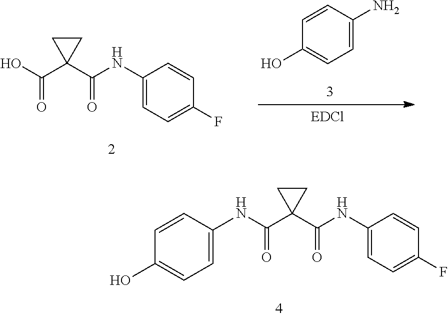 [SMILES: O=C(O)C1(C(=O)Nc2ccc(F)cc2)CC1 ; Nc1ccc(O)cc1 ; O=C(Nc1ccc(O)cc1)C1(C(=O)Nc2ccc(F)cc2)CC1]

To a solution of Compound 2 (10 g, 44.80 mmol, 1 eq.) and Compound 3 (5.87 g, 53.8 mmol, 1.2 eq.) in dimethyl acetamide (DMA) (60 mL) was added 3-(ethyliminomethyleneamino)-N,N-dimethyl-propan-1-amine hydrochloride (EDCI) (10.31 g, 53.8 mmol, 1.2 eq.). The mixture was stirred vigorously at 20° C. until the reaction was complete. The mixture was poured into aqueous (aq) saturated NaHCO₃ (400 mL) and extracted with EtOAc (4×100 mL). The combined organic phases were washed with aqueous saturated NaCl (100 mL), dried over anhydrous (anhyd) Na₂SO₄, and concentrated. Compound 4 (21 g, crude) (50% purity) was obtained. ¹H NMR (400 MHz, DMSO-d₆) δ 10.16 (br s, 1H), 9.72 (br s, 1H), 7.61 (dd, 2H), 7.34 (d, 2H), 7.13 (t, 2H) 6.68 (d, 2H), 1.42 (s, 4H); MS (EI) for C₁₇H₁₅FN₂O₃, found 314.9 (MH+).

### Step 2: Methyl 4-[4-[[1-[(4-fluorophenyl)carbamoyl]cyclopropane-carbonyl]amino]phenoxy]-7-methoxyquinoline-6-carboxylate (6)

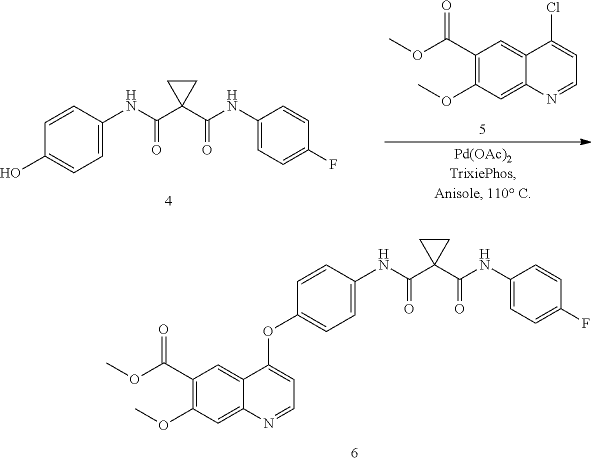 [SMILES: O=C(Nc1ccc(O)cc1)C1(C(=O)Nc2ccc(F)cc2)CC1 ; COC(=O)c1cc2c(Cl)ccnc2cc1OC ; COC(=O)c1cc2c(Oc3ccc(NC(=O)C4(C(=O)Nc5ccc(F)cc5)CC4)cc3)ccnc2cc1OC]

A mixture of Compound 4 (5.99 g, 9.5 mmol, 1.2 eq.), Compound 5 (2 g, 8.0 mmol, 1.0 eq.), Pd(OAc)₂ (89 mg, 397.4 μmol, 0.05 eq.), rac-2-(Di-tert-butylphosphino)-1,1′-binaphthyl (TrixiePhos, 316.71 mg, 794.7 μmol, 0.1 eq.) and K₃PO₄ (2.53 g, 11.9 mmol, 1.5 eq.) in anisole (50 mL) was stirred at 110° C. for 2 hours (h) under an atmosphere of nitrogen. The mixture was filtered, and the filtrate was concentrated. The residue was purified by flash silica gel chromatography (1:1 petroleum ether:EtOAc to 20:1 EtOAc:MeOH). Compound 6 was obtained (2.6 g, 61.8% yield). ¹H NMR (400 MHz, CDCl₃) δ 9.38 (s, 1H), 8.80 (s, 1H), 8.63 (d, 2H), 7.64 (d, 2H), 7.54-7.41 (m, 3H), 7.18 (d, 2H), 7.09-7.01 (m, 2H), 6.43 (d, 1H), 4.05 (s, 3H), 3.97 (s, 3H), 1.78-1.72 (m, 2H), 1.69-1.63 (m, 2H); MS (EI) for C₂₉H₂₄FN₃O₆, found 530.0 (MH+).

### Step 3: 4-[4-[[1-[(4-Fluorophenyl)carbamoyl]cyclopropane-carbonyl]amino]phenoxy]-7-methoxyquinoline-6-carboxylic acid (7)

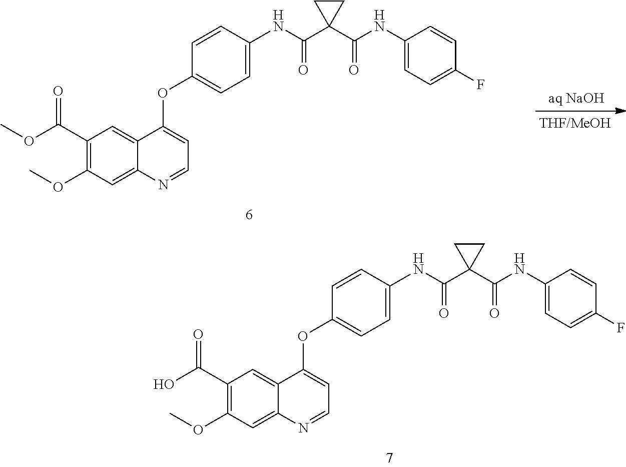 [SMILES: COC(=O)c1cc2c(Oc3ccc(NC(=O)C4(C(=O)Nc5ccc(F)cc5)CC4)cc3)ccnc2cc1OC ; COc1cc2nccc(Oc3ccc(NC(=O)C4(C(=O)Nc5ccc(F)cc5)CC4)cc3)c2cc1C(=O)O]

To a solution of Compound 6 (1.8 g, 3.4 mmol, 1 eq.) in tetrahydrofuran (THF) (15 mL) and MeOH (15 mL) was added 2 M aqueous NaOH (7 mL, 4.1 eq.). The mixture was stirred at 6-13° C. for 4 hours. The mixture was adjusted to a pH of approximately 8 with 1 M aqueous HCl and concentrated to remove solvent. Water (50 mL) was added, and the mixture was adjusted to a pH of approximately 6 with 1 M aqueous HCl. The resulting precipitate was filtered, washed with water (2×10 mL), and dried under vacuum. Compound 7 was obtained (1.7 g, 97.0% yield). ¹H NMR (400 MHz, DMSO-d₆) δ 10.22 (s, 1H), 10.08 (s, 1H), 8.65 (d, 1H), 8.48 (s, 1H), 7.77 (d, 2H), 7.64 (dd, 2H) 7.47 (s, 1H), 7.25 (d, 2H), 7.15 (t, 2H), 6.45 (d, 1H), 3.96 (s, 3H), 1.47 (s, 4H); MS (EI) for C₂₈H₂₂FN₃O₆, found 516.1 (MH+).

### Step 4: 1-N′-(4-Fluorophenyl)-1-N-[4-[7-methoxy-6-(methylcarbamoyl)quinolin-4-yl]oxyphenyl]cyclopropane-1,1-dicarboxamide (1)

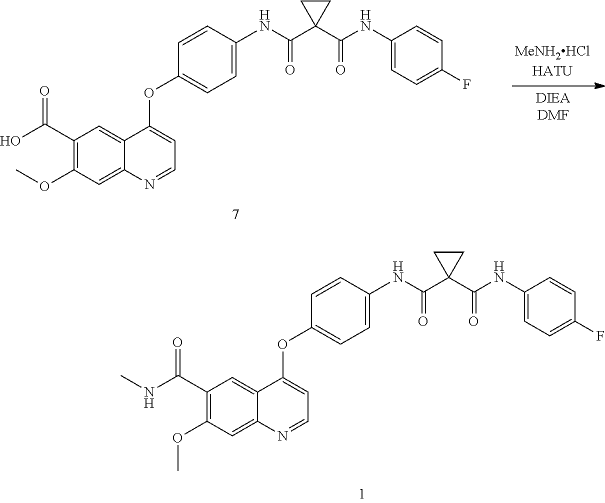 [SMILES: COc1cc2nccc(Oc3ccc(NC(=O)C4(C(=O)Nc5ccc(F)cc5)CC4)cc3)c2cc1C(=O)O ; CNC(=O)c1cc2c(Oc3ccc(NC(=O)C4(C(=O)Nc5ccc(F)cc5)CC4)cc3)ccnc2cc1OC]

A solution of Compound 7 (300 mg, 582.0 μmol, 1 eq.), HATU (332 mg, 873.2 mol, 1.5 eq.), and DIEA (301 mg, 2.3 mmol, 406 μL, 4 eq.) in DMF (10 mL) was stirred at 6-10° C. for 1 hour. Methanamine hydrochloride (79 mg, 1.2 mmol, 2.0 eq.) was added, and the mixture was stirred at 6-10° C. for 17 hours. The mixture was filtered, and the resulting filtrate purified by prep HPLC (Column: Waters Xbridge 150 mm*25 mm*5 m, gradient: 33-63% of acetonitrile in 10 mM aqueous NH₄HCO₃, flow rate: 25 mL/min). Compound 1 was obtained (105.4 mg, 34.3% yield). 1H NMR (400 MHz, DMSO-d₆) δ 10.20 (s, 1H), 10.06 (s, 1H), 8.65 (d, 1H), 8.61 (s, 1H), 8.42-8.33 (m, 1H), 7.77 (d, 2H), 7.68-7.61 (m, 2H), 7.51 (s, 1H), 7.25 (d, 2H), 7.19-7.11 (m, 2H), 6.46 (d, 1H), 4.02 (s, 3H), 2.84 (d, 3H) 1.47 (s, 4H); MS (EI) for C₂₉H₂₅FN₄O₅, found 529.1 (MH+).

### Example 1: Preparation of Compound 1 Fumarate Form A

Fumaric acid (1 eq.) in acetone was added to the free base of Compound 1 (1 eq.) and the resulting reddish slurry was stirred at about 50° C. for 4 days. The slurry was then SC to RT and stirred for an addition 1 day to provide a pink slurry. The solids were then removed by positive pressure filtration to provide a mixture of Fumarate Form A and free base Form A.

### Example 2: Preparation of Compound 1 Hemifumarate Form B

Fumaric acid (2 eq.) in acetone was added to the free base of Compound 1 (1 eq.) and the resulting reddish slurry was stirred at about 50° C. for 6 days to provide a resulting off-white slurry. The solids were then removed by positive pressure filtration of the hot solution to provide Hemifumarate Form B.

### Example 3: Preparation of Compound 1 HCl Form A

1 eq. of HCl was added to the free base of Compound 1 in THF and the resulting dark reddish slurry was stirred at RT for 3 days to provide a resulting thick off-white slurry. The solids were then removed by positive pressure filtration to provide HCl Form A.

### Example 4: Preparation of Compound 1 HCl Form B

1 eq. of HCl was added to the free base of Compound 1 in chloroform and the resulting reddish slurry was stirred at about 50° C. for 3 days to provide a resulting pale pink slurry. The solids were then removed by positive pressure filtration to provide HCl Form B.

### Example 5: Preparation of Compound 1 HCl Form C

1 eq. of HCl was added to the free base of Compound 1 in methanol at a temperature of about 60° C. resulting in a yellowish slurry. The solution was then CC to about −20° C. and kept cold for about 2 days to provide a clear orange solution. Partial FE provided a clear red solution and then four volumes of the anti-solvent MTBE was added and the solution was stirred for 1 day at RT to provide off-white solid Compound 1 HCl Form C that was separated by positive pressure filtration.

### Example 6: Preparation of Compound 1 HCl Form D

2 eq. HCl was added to the free base of Compound 1 at about 50° C., and the resulting pink slurry was stirred at 50° C. for 5 days. The solid Compound 1 HCl Form D was separated by positive pressure filtration.

### Example 7: Preparation of Compound 1 Form A

Compound 1 Form A is likely the most thermodynamically stable crystalline form of the free base of Compound 1. Accordingly, multiple procedures lead to the formation of this form. A list of some of the possible procedures to obtain Compound 1 Form A are listed in Table 1. This list in Table 1 is not meant to be exclusive, indeed there are likely many more procedures that will produce this form.

TABLE 1
Selected procedures for producing Compound 1 Form A

| Solvent | Conditions |
|---|---|
| ACN/water 80:20 | 1) Slurry at 2-8° C. for 14 d; or |
| | 2) Slurry at room temperature for 14 d |
| Chloroform | Slurry at 57° C. for 2 days |
| DCM | Slurry at room temperature for 14 days |
| Ethyl Acetate | Slurry at 76° C. for 3 days |
| Ethanol | 1) Slurry at room temperature for 14 days; |
| | or |
| | 2) Slurry at 76° C. for 3 days |
| Ethanol/water 90:10 | Slurry at room temperature for 14 days |
| Isopropyl alcohol | 1) Slurry at room temperature for 14 days; |
| | or |
| | 2) Slurry at 76° C. for 3 days |
| Methanol | 1) Slurry at room temperature for 14 days; |
| | 2) Slurry at 57-58° C. for 4 days; or |
| | 3) Fast evaporation |
| Methanol/Ethyl Acetate 3:2 | Slurry at room temperature for 14 days |
| 2,2,2-Trifluoroethanol | 1) Slow evaporation; |
| | 2) Fast evaporation; or |
| | 3) Crash precipitation using diethyl ether |
| | as the anti-solvent, then slurry for 1 day. |
| Tetrahydrofuran | 1) Slurry at room temperature for 14 days; |
| | or |
| | 2) Slurry at 57-58° C. for 4 days |
| Tetrahydrofuran/water 50:50 | Slurry at room temperature for 14 days |

### Example 8: Preparation of Compound 1 Form B

Compound 1 was dissolved in AcOH, and crystallized by VD with diethyl ether as the anti-solvent.

### Example 9: Preparation of Compound 1 Form C

Compound 1 was dissolved in HFIPA, and crystallized by CP with MTBE as the anti-solvent.

### Example 10: Preparation of Compound 1 Form D

Compound 1 was dissolved in methanol, and crystallized by CC. The mixture was then slurried at 2-8° C. to provide Form D.

### Example 11: Preparation of Compound 1 Form E

Method A: Compound 1 was dissolved in THF, and crystallized by CC.

Method B: Compound 1 was dissolved in 90:10 THF:Water, and precipitated by CP.

### Example 12: Preparation of Compound 1 Form F

Method A: Compound 1 was dissolved in chloroform, and crystallized by SE.

Method B: Compound 1 was slurried in chloroform.

### Example 13: Preparation of Compound 1 Form G

Compound 1 was dissolved in chloroform, and crystallized by placing the mixture in the freezer.

### Example 14: Preparation of Compound 1 Form H

Form H was obtained by VS of Amorphous Compound 1 with DCM.

### Example 15: Preparation of Compound 1 Form K

Compound 1 Form K was made by desolvation of Form F or Form G, which are chloroform solvates.

### Example 16: Preparation of Compound 1 Form O

Compound 1 Form 0 was discovered during salt attempts with various counterions in TFE-containing solvent systems, and is likely a TFE solvate.

### Example 17: Preparation of Compound 1 Phosphate Form A

1 molar equivalent of phosphoric acid was added to a slurry of Compound 1 in chloroform, and then the resulting mixture was slurried for 3 days at about ˜50° C. The product was isolated by positive pressure filtration.

### Example 18: Preparation of Compound 1 Form I

Compound 1 in a 90:10 THF/water mixture was crash precipitated with heptane and then stirred at freezing temperatures for 7 days.

### Example 19: Preparation of Compound 1 Form J

Compound 1 was slurried in acetone for 14 days.

### Example 20: Preparation of Compound 1 Form L

Compound 1 was slurried in chloroform for 14 days.

### Example 21: Preparation of Compound 1 Form M

Dehydration of Compound 1 Form E in a vacuum oven at ˜77° C. for 1 day.

### Example 22: Preparation of Compound 1 Form N

Compound 1 was slurried in a 70:30 mixture of TFE/MTBE for 7 days at room temperature.

### Preparative Example 2: Synthesis of Compound 1 Hemifumarate

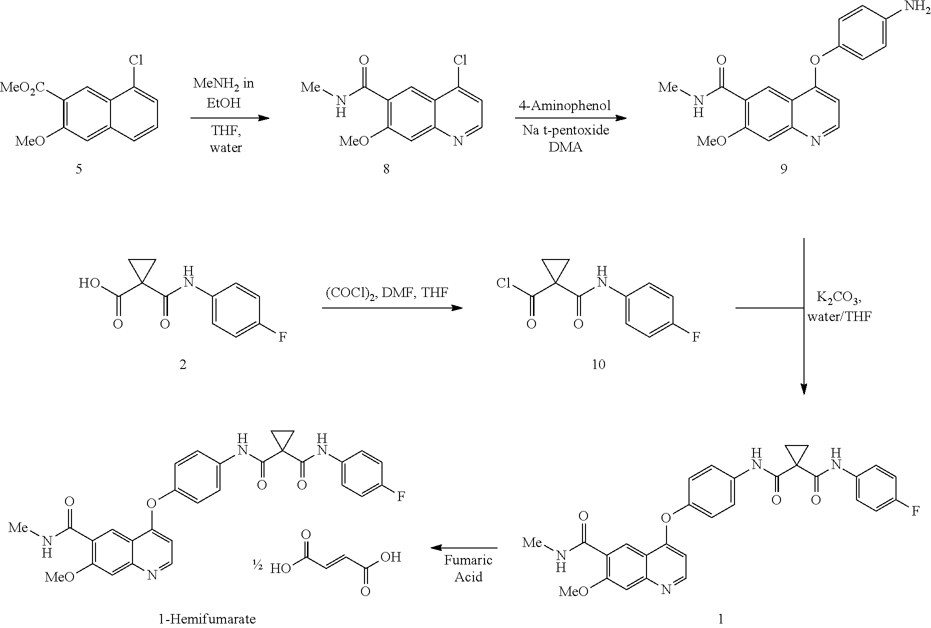

### Synthesis of 4-chloro-7-methoxy-N-methylquinoline-6-carboxamide

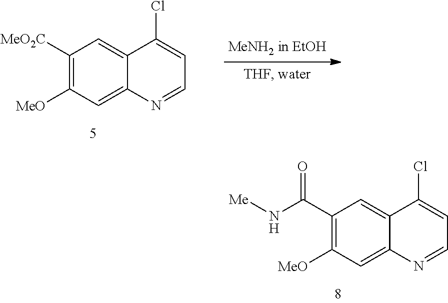 [SMILES: COC(=O)c1cc2c(Cl)ccnc2cc1OC ; CNC(=O)c1cc2c(Cl)ccnc2cc1OC]

To a suspension of methyl 4-chloro-7-methoxyquinoline-6-carboxylate 5 (2 g, 8 mmol) in THF (20 mL) was added methyl amine in EtOH (33% w/w, 8 M, 20 mL, 160 mmol) and H₂O (10 mL). The resulting mixture was stirred at room temperature. The mixture turned into a clear solution in about 10 min and remained as a clear solution during the reaction. The stirring was continued until the starting material was completely consumed as evidenced by LCMS and HPLC. It took about 3 hours. The mixture was then concentrated and the residue was slurried in 20 mL of water, and filtered. Some EtOAc was used to transfer the material from flask to filter funnel. The product was dried to give 4-chloro-7-methoxy-N-methylquinoline-6-carboxamide as white solid (yield 1.8 g, 90%, HPLC purity >97%).

### Synthesis of 4-(4-aminophenoxy)-7-methoxy-N-methylquinoline-6-carboxamide

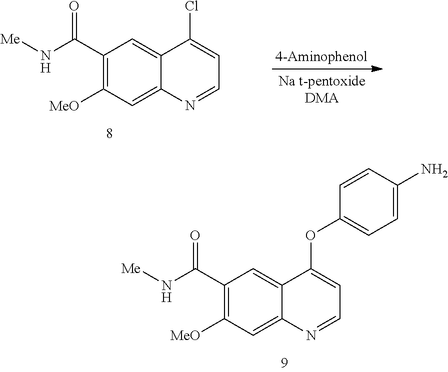 [SMILES: CNC(=O)c1cc2c(Cl)ccnc2cc1OC ; CNC(=O)c1cc2c(Oc3ccc(N)cc3)ccnc2cc1OC]

A 5 L, 3-neck round bottom flask equipped with a thermometer, nitrogen inlet, and magnetic stirrer was charged with 4-chloro-7-methoxy-N-methylquinoline-6-carboxamide (3; 300 g; 1 eq.), 4-aminophenol (195.9 g; 1.5 eq.), and DMA (1500 mL). The resulting solution was stirred at room temperature, and a solution of sodium t-pentoxide (184.52 g; 1.4 eq.) dissolved in anhydrous THF (313 mL) was added with stirring over a 5 minute period. The reaction mixture was then heated to 75-80° C. and stirred for an additional 2-6 hours. The reaction mixture was then cooled to room temperature and charged with water (3 L), and stirred at least for an additional 1 hour. The product was filtered and washed twice with 600 mL of 1:1 DMA/water, then once with 1200 mL water. The product was transferred to a crystallizing dish and dried in the vacuum oven at 40-45° C. for a minimum of 18 hours to yield a light brown shiny solid (370-377 g; 96-97%).

### Synthesis of 1-((4-fluorophenyl)carbamoyl)cyclopropane-1-carbonyl chloride

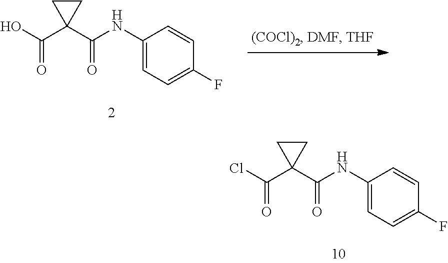 [SMILES: O=C(O)C1(C(=O)Nc2ccc(F)cc2)CC1 ; O=C(Cl)C1(C(=O)Nc2ccc(F)cc2)CC1]

A 250 mL, 3 neck round bottom flask equipped with a thermometer, nitrogen inlet, and magnetic stirrer was charged with 1-((4-fluorophenyl)carbamoyl)cyclopropane-1-carboxylic acid (2, 19.11 g; 1.3 eq.), 75 mL anhydrous THF, and 0.25 mL DMF (catalyst). The mixture was stirred until all solids dissolved, cooled to 5-10° C., and then charged with oxalyl chloride (7.13 mL; 1.28 eq.). The resulting mixture was aged at 10-15° C. for 2-3 hours and reaction completion was confirmed by IPC (in process control). Upon reaction completion, the resulting product mixture was used in the next step without further purification.

### Alternative Synthesis of 1-((4-fluorophenyl)carbamoyl)cyclopropane-1-carbonyl chloride

A 250 mL, 3 neck round bottom flask equipped with a thermometer, nitrogen inlet, and magnetic stirrer was charged with 1-((4-fluorophenyl)carbamoyl)cyclopropane-1-carboxylic acid (2, 19.11 g; 1.3 eq.), 75 mL anhydrous THF, and 0.25 mL DMF (catalyst). The mixture was stirred until all solids dissolved, cooled to 5-15° C., and then charged with oxalyl chloride (7.13 mL; 1.28 eq.). The resulting mixture was warmed to room temperature and then stirred for 2-4 hours. The resulting product mixture was used in the next step without further purification.

### Synthesis of N-(4-fluorophenyl)-N-(4-((7-methoxy-6-(methylcarbamoyl)quinolin-4-yl)oxy)phenyl)cyclopropane-1,1-dicarboxamide (1)

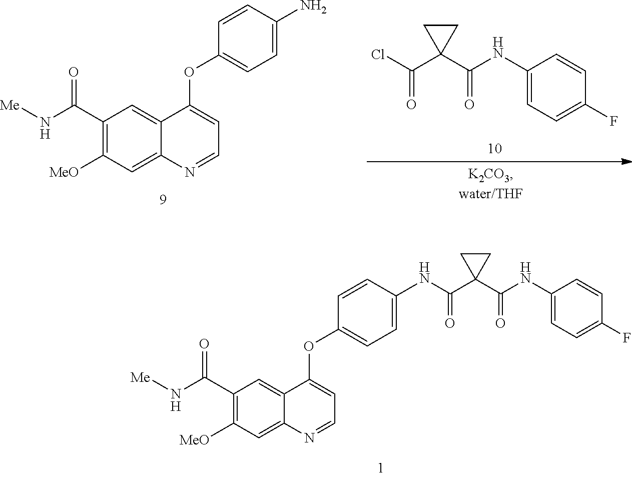 [SMILES: CNC(=O)c1cc2c(Oc3ccc(N)cc3)ccnc2cc1OC ; O=C(Cl)C1(C(=O)Nc2ccc(F)cc2)CC1 ; CNC(=O)c1cc2c(Oc3ccc(NC(=O)C4(C(=O)Nc5ccc(F)cc5)CC4)cc3)ccnc2cc1OC]

A 500 mL, 3 neck round bottom flask equipped with a thermometer, nitrogen inlet, and magnetic stirrer was charged with 4-(4-aminophenoxy)-7-methoxy-N-methylquinoline-6-carboxamide (9, 21.3 g; 1.0 eq.), 210 mL anhydrous THF, and a solution composed of potassium carbonate (27.32 g; 3 eq) and 100 mL water. The added aqueous K₂CO₃ solution was rinsed forward with an additional 6.4 mL water. With vigorous agitation, the reaction mixture containing Compound 10 from the previous example was transferred to the present reaction mixture over a period of no less than 30 minutes while maintaining an internal temperature between 20 and 25° C. The transfer equipment was rinsed with 32 mL of anhydrous THF. The reaction mixture was agitated at ambient temperature for 0.5-1 hour. The resulting mixture was warmed to 35-40° C. and the phases were allowed to separate. The lower aqueous layer was discarded and the top organic phase was warmed to 55-60° C. and then polish filtered and rinsed with 21 mL of THF. The filtered organic phase was transferred to a 1 L, 3 neck round bottom flask equipped with thermometer, nitrogen inlet, and mechanical stirring and charged, with water at 55-60° C. The resulting solution was seeded with Compound 1 and to the resulting seed bed, water was added as an anti-solvent over 4-4.5 hours while maintaining a temperature of 50-55° C. The resulting slurry was cooled to 20-25° C. and aged for no less than 2 hours. The product was then filtered, washed with water/THF and dried.

### Alternative Synthesis of N-(4-fluorophenyl)-N-(4-((7-methoxy-6-(methylcarbamoyl)quinolin-4-yl)oxy)phenyl)cyclopropane-1,1-dicarboxamide (1)

A 500 mL, 3 neck round bottom flask equipped with a thermometer, nitrogen inlet, and magnetic stirrer was charged with 4-(4-aminophenoxy)-7-methoxy-N-methylquinoline-6-carboxamide (9, 21.3 g; 1.0 eq.), 210 mL anhydrous THF, and a solution composed of potassium carbonate (27.32 g; 3 eq) and 100 mL water. The added aqueous K₂CO₃ solution was rinsed forward with an additional 6.4 mL water. With vigorous agitation, the reaction mixture containing Compound 10 from the previous example was transferred to the present reaction mixture over a period of 0.5-1 hour while maintaining an internal temperature below 27° C. The transfer equipment was rinsed with 32 mL of anhydrous THF. The reaction mixture was agitated at ambient temperature for 0.5-1 hour. The resulting mixture was warmed to 35-40° C. and the phases was allowed to separate. The lower aqueous layer was discarded and the top organic phase was warmed to 45-50° C. and then filtered through a filter paper and rinsed with 21 mL of THF. The filtered organic phase was transferred to a 1 L, 3 neck round bottom flask equipped with thermometer, nitrogen inlet, and mechanical stirring and charged, over a minimum of 1 hour with 694 mL of filtered water. The resulting mixture was stirred at 20-25° C. for a minimum of 12 hours, and the product was then filtered and rinsed twice with 42 mL of a 2:1 water:THF mixture. The product was then dried on a filter paper at room temperature or in a vacuum oven at 40-45° C. to yield a white to beige solid (31.36 g; 90%).

### Synthesis of N-(4-fluorophenyl)-N-(4-((7-methoxy-6-(methylcarbamoyl)quinolin-4-yl)oxy)phenyl)cyclopropane-1,1-dicarboxamide·1/2 fumaric acid (1·hemifumarate)—Method 1

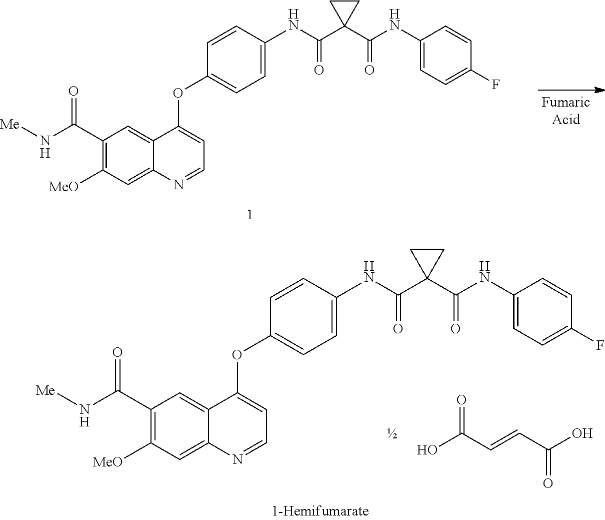 [SMILES: CNC(=O)c1cc2c(Oc3ccc(NC(=O)C4(C(=O)Nc5ccc(F)cc5)CC4)cc3)ccnc2cc1OC ; CNC(=O)c1cc2c(Oc3ccc(NC(=O)C4(C(=O)Nc5ccc(F)cc5)CC4)cc3)ccnc2cc1OC ; O=C(O)/C=C/C(=O)O]

A 2000 mL, 3 neck round bottom flask equipped with a thermometer, nitrogen inlet, and magnetic stirrer was charged with fumaric acid (80 g; 0.82 eq.) and 1.2 L of a 20% solution of water in ethanol. The mixture was heated to 45-50° C. and stirred until all solids were dissolved. To a separate 3 L, 3 neck round bottom flask equipped with thermometer, nitrogen inlet, and mechanical stirrer was charged N-(4-fluorophenyl)-N-(4-((7-methoxy-6-(methylcarbamoyl)quinolin-4-yl)oxy)phenyl)cyclopropane-1,1-dicarboxamide (1, 500 g; 1.0 eq.). The fumaric acid solution was clarified through a filter paper at 40-45° C., and transferred, at 40-45° C., to the flask with Compound 1. The 2000 mL round bottom flask was rinsed forward with 300 mL of a 20% solution of water in ethanol at 45-50° C. The resulting mixture was heated to reflux (75-80° C.) and stirred for 4-6 hours. The reaction mixture was then cooled to room temperature, and the product was filtered and the filter cake was washed twice with 300 mL of a 20% solution of water in ethanol. The product was then dried on a filter paper at room temperature or in a vacuum oven at 40-45° C. to yield a white to beige solid (472-474 g; 97%).

### Synthesis of N-(4-fluorophenyl)-N-(4-((7-methoxy-6-(methylcarbamoyl)quinolin-4-yl)oxy)phenyl)cyclopropane-1,1-dicarboxamide-12 fumaric acid (1·hemifumarate)—Method 2

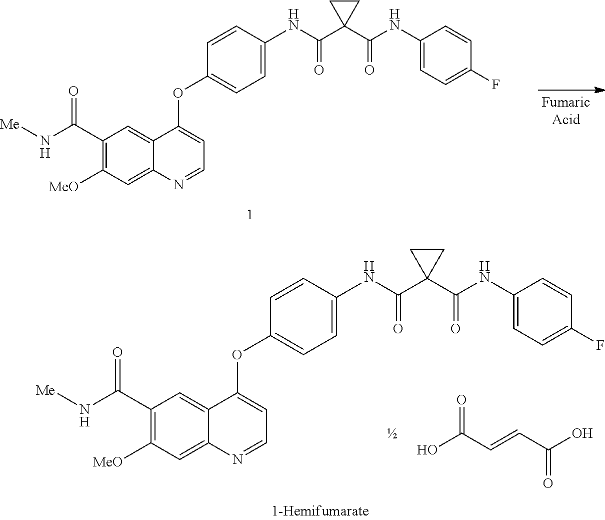 [SMILES: CNC(=O)c1cc2c(Oc3ccc(NC(=O)C4(C(=O)Nc5ccc(F)cc5)CC4)cc3)ccnc2cc1OC ; CNC(=O)c1cc2c(Oc3ccc(NC(=O)C4(C(=O)Nc5ccc(F)cc5)CC4)cc3)ccnc2cc1OC ; O=C(O)/C=C/C(=O)O]

Fumaric acid (2.68 g, 1 eq.) and EtOH/acetone, 1:1 (48 mL) were added to a two-piece EasyMax (EM) reaction vessel, and heated to a reaction temperature of 50° C. to dissolve all material. In the adjacent EM pot, a 1-piece EM vessel containing Compound 1 (12.0 g, 1 eq.) was set to a jacket temperature of 50° C. The fumaric acid solution was transferred to the vessel containing Compound 1. Seed was charged (2% seed, 0.244 g), and the vessel was heated to reflux (−65° C.). After 1 hour, 0.5 mL of the slurry was filtered, washed with EtOH (6×1.5 mL) and analysed by HPLC to determine fumaric acid content (result should be about 10%). The slurry was then cooled to 25° C. over 1 hour and stirred for a further 1 hour. The solids were then filtered, washed with 1:1 EtOH/acetone (2×3 V), and dried over the weekend at 25° C. under vacuum. ¹H NMR 700 MHz (DMSO-d₆) δ 1.473 (s, 4H), δ 4.009 (s, 3H), δ 2.839 (d, 3H, ³J_{1H-1H}=4.7 Hz), δ 2.840 (d, 3H, ³J_{1H-1H}=4.7 Hz), δ 6.450 (d, 1H, ³J_{1H-1H}=5.2 Hz), δ 6.632 (s, 2H), δ 6.635 (s, 2H), δ 7.137 (m, 2H), δ 7.244 (d, 2H, ³J_{1H-1H}=8.6 Hz), δ 7.494 (s, 1H), δ 7.642 (m, 2H), δ 7.776 (d, 2H. ³J_{1H-1H}=8.6 Hz), δ 8.361 (q, 1H, ³J_{1H-1H}=4.7 Hz), δ 8.618 (s, 1H), 8.615 (s, 1H), δ 8.638 (d, 1H, ³J_{1H-1H}=5.2 Hz), δ 10.070 (s, 1H), δ 10.216 (s, 1H), δ 13.164 (s, 1H). ¹⁹F NMR 700 MHz (DMSO-d₆; ref trifluorotoluene at −63.72 ppm) δ −121.460. ¹³C NMR 700 MHz (DMSO-d₆) δ 15.46, δ 26.47, δ 31.60, δ 56.15, δ 102.91, δ 107.83, δ 114.55, δ 115.05 (d, ²J_{19F-13C}=22.2 Hz), δ 121.15, δ 122.23, δ 122.43 (d, ³J_{19F-13C}=7.6 Hz), δ 124.35, δ 125.24, δ 134.03, δ 135.22 (d, ⁴J_{19F-13C}=2.4 Hz), δ 136.73, δ 149.08, δ 151.46, δ 153.18, δ 157.94, δ 158.30 (d, ¹J_{19F-13C}=240.2 Hz), δ 161.76, δ 164.89, δ 168.16, and δ 168.16. ¹⁵N NMR 700 MHz (DMSO-d₆) δ 106.25 (¹⁵N), δ 127.79 (¹⁵N), δ 128.86 (¹⁵N), δ 166.04, δ 289.56 (¹⁵N).

### Pharmaceutical Composition Examples

Compound 1 was designed as a solid oral tablet dosage form with 20 mg strength. The initial tablets contained a 25% drug load formulation (freebase equivalent). Each tablet consisted of a granulated blend of drug substance with microcrystalline cellulose, lactose anhydrous, hydroxypropyl cellulose, croscarmellose sodium, colloidal silicon dioxide, and magnesium stearate. This tablet formulation was designated Compound 1 Pharmaceutical Composition A. Compound 1 Pharmaceutical Composition A tablets were coated with an Opadry® II Blue (85F105057) (Colorcon, West Point, PA) film coating system. The list of excipients and their functions in Pharmaceutical Composition A are presented in the following table.

TABLE 2
Compound 1 Pharmaceutical Composition A

| Ingredient | Function |
|---|---|
| Compound 1 | Active ingredient |
| Microcrystalline Cellulose, PH-102 | Diluent |
| Lactose Anhydrous, 60M | Diluent |
| Hydroxypropyl Cellulose, EXF | Binder |
| Croscarmellose Sodium | Disintegrant |
| Colloidal Silicon Dioxide | Glidant |
| Magnesium Stearate (Non-Bovine) | Lubricant |
| Opadry ® II Blue (85F105057) | Film coating |

Preparation of Compound 1 Pharmaceutical Composition A tablets (20 mg) includes delumping excipients, followed by high-shear granulation, delumping of wet granules, fluid bed drying, dry milling, extra-granular blending, lubrication blending, tableting, film coating, and packaging.

Thus, microcrystalline cellulose PH1102, lactose anhydrous 60M, hydroxypropyl cellulose EXF, and croscarmellose sodium were passed through a 20 mesh screen. Compound 1 was added, and the mixture was placed in a high shear granulation bowl and high shear granulated with purified water. The wet granules were then passed through a Comil or were hand screened. The wet granules were then dried using a fluid bed dryer and then passed through a Comil. The milled granules were then charged to a blender along with delumped colloidal silicon dioxide and croscarmellose sodium and the mixture was blended. Magnesium stearate (non-bovine) that had been passed through a 30 mesh screen was then added to this mixture as blending was continued. The lubricated blend was then compressed using an instrumented rotary tablet press. The coating suspension was then prepared by adding Opadry® II Blue to purified water to provide a dispersion. The dispersion was slowly sprayed onto the core tablets loaded into a perforated pan coater. The coated tablets were packaged in child resistant HDPE bottles along with desiccants and a polyester coil.

Compound 1 Pharmaceutical Composition B provided improved manufacturing processing and increased manufacturing efficiency with the same drug loading. The manufacturing process for Compound 1 Pharmaceutical Composition A were required frequent stops for machine cleaning because of sticking issues, which may result in defective tablets. Compound 1 Pharmaceutical Composition B eliminated the need for frequent stopping and cleaning. By changing the lubricant from magnesium stearate to stearic acid and increasing the binder concentration, for example, from 3% hydroxypropyl cellulose to 5% hydroxypropyl cellulose in the formulation, the sticking problem was avoided. Thus, Compound 1 Pharmaceutical Composition B can be produced continuously, increasing the efficiency and meeting the scale-up demand.

Each Compound 1 Pharmaceutical Composition B tablet consisted of a granulated blend of drug substance with microcrystalline cellulose, lactose anhydrous, hydroxypropyl cellulose, croscarmellose sodium, colloidal silicon dioxide, and stearic acid. The tablets were coated with an Opadry® II Blue (85F105057) film coating system.

An 80 mg dose strength tablet of Pharmaceutical Composition B was prepared to enable higher doses to be administered with fewer tablets; 100 mg and 120 mg dose strength tablets of Pharmaceutical Composition B were also prepared. The Pharmaceutical Composition B tablet strengths were prepared from a common blend and were film-coated. The tablet dose strengths were distinguished by shape, with the 20 mg tablets and the 80 mg tablets being round and oval, respectively. The list of excipients and their functions in Pharmaceutical Composition B are presented in the following table.

TABLE 3
Compound 1 Pharmaceutical Composition B

| Ingredient | Function |
|---|---|
| Compound 1 | Active ingredient |
| Microcrystalline Cellulose, PH-102 | Diluent |
| Lactose Anhydrous, 60M | Diluent |
| Hydroxypropyl Cellulose, EXF | Binder |
| Croscarmellose Sodium | Disintegrant |
| Colloidal Silicon Dioxide | Glidant |
| Stearic Acid 50 (Vegetable Grade) | Lubricant |
| Opadry ® II Blue (85F105057) | Film coating |

Preparation of Compound 1 Pharmaceutical Composition B tablets (20 mg and 80 mg) consisted of delumping excipients, followed by high-shear granulation, delumping of wet granules, fluid bed drying, dry milling, extra-granular blending, lubrication blending, tableting, film coating, and packaging.

Thus, microcrystalline cellulose anhydrous PH102, anhydrous lactose, hydroxypropyl cellulose EXF, and croscarmellose sodium were passed through a 20 mesh screen. A binding solution was separately prepared by adding hydroxypropyl cellulose and purified water. Compound 1, the screened mixture of microcrystalline cellulose anhydrous PH102, anhydrous lactose, hydroxypropyl cellulose EXF, and croscarmellose sodium were high shear granulated in a high shear granulation bowl along with the binder solution. The resulting wet granules were passed through a Comil or hand screened, dried using a fluid bed dryer, and passed through a Comil. The milled granules were then combined with colloidal silicon dioxide and croscarmellose sodium and blended in a blender. Stearic acid that had been passed through a 30 mesh screen was then charged into the blender. The lubricated blend was then compressed using an instrumented rotary tablet press. The coating suspension was then prepared by adding Opadry® II Blue to purified water to provide a dispersion. The dispersion was slowly sprayed onto the core tablets loaded into a perforated pan coater. The coated tablets were packaged in child resistant HDPE bottles along with dessicants and a polyester coil.

The quantitative unit compositions of the Compound 1 tablets that were investigated are presented in the following tables where Compound 1 is present as the free base, including Forms A, B, C, D, D, E, F, G, H, K, O, or Q disclosed herein. The compositions can also accommodate salt forms of Compound 1, including the HCl, fumaric acid, and phosphoric acid salts disclosed herein, including HCl salt Forms A, B, C, and D; fumaric acid Form A; hemifumarate Form B; and phosphoric acid Form A. The amount of the Compound 1 salt that is used is adjusted to provide 20 mg, 40 mg, 60 mg, 80 mg, 100 mg, or 120 mg of Compound 1 (freebase equivalent).

TABLE 4
Compound 1 Pharmaceutical Composition A

Composition

| Ingredient | % w/w | mg/unit dose |
|---|---|---|
| Compound 1 | 27.75 | 20¹ |
| Microcrystalline Cellulose, PH-102 | 41.47 | 33.17 |
| Lactose Anhydrous, 60M | 20.73 | 16.59 |
| Hydroxypropyl Cellulose, EXF | 3.00 | 2.40 |
| Croscarmellose Sodium | 6.00 | 4.80 |
| Colloidal Silicon Dioxide | 0.30 | 0.24 |
| Magnesium Stearate (Non-Bovine) | 0.75 | 0.60 |
| Total core tablet weight | | 80.00 |
| Opadry ® II Blue (85F105057) | 4.00 | 3.20 |
| Total coated tablet weight | | 83.20 |

¹20 mg of Compound 1 free base is equivalent to 22.20 mg of Compound 1 hemifumarate salt.

TABLE 5
Composition of Compound 1 Tablets, 20 mg, 40 mg, 60 mg,
80 mg, 100 mg and 120 mg (Pharmaceutical Composition A)

| | Composition | | | | | | |
|---|---|---|---|---|---|---|---|
| | % | mg/unit dose | | | | | |
| Ingredient | w/w | 20 mg | 40 mg | 60 mg | 80 mg | 100 mg | 120 mg |
| Compound 1 | 27.75 | 20¹ | 40² | 60³ | 80⁴ | 100⁵ | 120⁶ |
| Microcrystalline | 41.47 | 33.17 | 66.34 | 99.51 | 132.68 | 165.85 | 199.02 |
| Cellulose, PH-102 | | | | | | | |
| Lactose Anhydrous, | 20.73 | 16.59 | 33.18 | 49.77 | 66.36 | 82.95 | 99.54 |
| 60M | | | | | | | |
| Hydroxypropyl | 3.00 | 2.40 | 4.80 | 7.20 | 9.60 | 12.0 | 14.4 |
| Cellulose, EXF | | | | | | | |
| Croscarmellose | 6.00 | 4.80 | 9.60 | 14.40 | 19.20 | 24.00 | 28.80 |
| Sodium | | | | | | | |
| Colloidal Silicon | 0.30 | 0.24 | 0.48 | 0.72 | 0.96 | 1.20 | 1.44 |
| Dioxide | | | | | | | |
| Magnesium Stearate | 0.75 | 0.60 | 1.20 | 1.80 | 2.40 | 3.00 | 3.60 |
| (Non-Bovine) | | | | | | | |
| Total core tablet | | 80.0 | 160.0 | 240.0 | 320.0 | 400.0 | 480.0 |
| weight | | | | | | | |
| Opadry ® II Blue | 4.00 | 3.20 | 6.40 | 9.60 | 12.80 | 16.00 | 19.20 |
| (85F105057) | | | | | | | |
| Total coated | | 83.2 | 166.4 | 249.6 | 332.8 | 416.0 | 499.2 |
| tablet weight | | | | | | | |

¹20 mg of Compound 1 free base is equivalent to 22.20 mg of Compound 1 hemifumarate salt.
²40 mg of Compound 1 free base is equivalent to 44.40 mg of Compound 1 hemifumarate salt.
³60 mg of Compound 1 free base is equivalent to 66.60 mg of Compound 1 hemifumarate salt.
⁴80 mg of Compound 1 free base is equivalent to 88.80 mg of Compound 1 hemifumarate salt.
⁵100 mg of Compound 1 free base is equivalent to 111.00 mg of Compound 1 hemifumarate salt.
⁶120 mg of Compound 1 free base is equivalent to 132.20 mg of Compound 1 hemifumarate salt.

TABLE 6
Composition of Compound 1 Tablets, 20 mg
and 80 mg (Pharmaceutical Composition B)

| | Composition | | |
|---|---|---|---|
| | | mg/unit dose | |
| Ingredient | % w/w | 20 mg | 80 mg |
| Compound 1 | 27.75 | 20¹ | 80² |
| Microcrystalline Cellulose, PH-102 | 38.63 | 30.90 | 123.62 |
| Lactose Anhydrous, 60M | 19.32 | 15.46 | 61.82 |
| Hydroxypropyl Cellulose, EXF | 5.00 | 4.00 | 16.00 |
| Croscarmellose Sodium | 6.00 | 4.80 | 19.20 |
| Colloidal Silicon Dioxide | 0.30 | 0.24 | 0.96 |
| Stearic Acid 50 | 3.00 | 2.40 | 9.60 |
| Total core tablet weight | | 80.0 | 320.0 |
| Opadry ® II Blue (85F105057) | 4.00 | 3.20 | 12.80 |
| Total coated tablet weight | | 83.2 | 332.8 |

¹20 mg of Compound 1 free base is equivalent to 22.20 mg of Compound 1 hemifumarate salt.
²80 mg of Compound 1 free base is equivalent to 88.78 mg of Compound 1 hemifumarate salt.

TABLE 7
Composition of Compound 1 Tablets, 20 mg, 40 mg, 60 mg,
80 mg, 100 mg and 120 mg (Pharmaceutical Composition B)

| | Composition | | | | | | |
|---|---|---|---|---|---|---|---|
| | % | mg/unit dose | | | | | |
| Ingredient | w/w | 20 mg | 40 mg | 60 mg | 80 mg | 100 mg | 120 mg |
| Compound 1 | 27.75 | 20¹ | 40² | 60³ | 80⁴ | 100⁵ | 120⁶ |
| Microcrystalline | 38.63 | 30.90 | 61.81 | 92.71 | 123.62 | 154.52 | 185.42 |
| Cellulose, PH-102 | | | | | | | |
| Lactose Anhydrous, | 19.32 | 15.46 | 30.91 | 46.37 | 61.82 | 77.28 | 92.74 |
| 60M | | | | | | | |
| Hydroxypropyl | 5.00 | 4.00 | 8.00 | 12.00 | 16.00 | 20.00 | 24.00 |
| Cellulose, EXF | | | | | | | |
| Croscarmellose | 6.00 | 4.80 | 9.60 | 14.40 | 19.20 | 24.00 | 28.80 |
| Sodium | | | | | | | |
| Colloidal Silicon | 0.30 | 0.24 | 0.48 | 0.72 | 0.96 | 1.20 | 1.44 |
| Dioxide | | | | | | | |
| Stearic Acid 50 | 3.00 | 2.40 | 4.80 | 7.20 | 9.60 | 12.00 | 14.40 |
| Total core | | 80.0 | 160.0 | 240.0 | 320.0 | 400.0 | 480.0 |
| tablet weight | | | | | | | |
| Opadry ® II Blue | 4.00 | 3.20 | 6.40 | 9.60 | 12.80 | 16.00 | 19.20 |
| (85F105057) | | | | | | | |
| Total coated | | 83.2 | 166.4 | 249.6 | 332.8 | 416.0 | 499.2 |
| tablet weight | | | | | | | |

¹20 mg of Compound 1 free base is equivalent to 22.20 mg of Compound 1 hemifumarate salt.
²40 mg of Compound 1 free base is equivalent to 44.40 mg of Compound 1 hemifumarate salt.
³60 mg of Compound 1 free base is equivalent to 66.60 mg of Compound 1 hemifumarate salt.
⁴80 mg of Compound 1 free base is equivalent to 88.80 mg of Compound 1 hemifumarate salt.
⁵100 mg of Compound 1 free base is equivalent to 111.00 mg of Compound 1 hemifumarate salt.
⁶120 mg of Compound 1 free base is equivalent to 132.20 mg of Compound 1 hemifumarate salt.

Stability. Pharmaceutical Composition B tablets containing Compound 1 hemifumarate salt B underwent stability testing. The coated tablets were packaged in child resistant HDPE bottles along with dessicants and a polyester coil. The coated tablets were subjected to long term stability testing conditions at 25° C. and 60% relative humidity (RH). When last checked at 12 months, the tablets exhibited less than 0.5 percent decomposition of Compound 1 as the hemifumarate salt Form B. The tablets were non-hygroscopic.

The coated tablets were packaged in child resistant HDPE bottles along with dessicants and a polyester coil. The coated tablets were subjected to accelerated stability testing conditions at 40° C. and 75% relative humidity (RH). When last checked at 6 months, the tablets exhibited less than 0.5 percent decomposition of Compound 1 as the hemifumarate salt Form B.

Dissolution. The Pharmaceutical Composition B tablets containing Compound 1 hemifumarate salt Form B from the stability tests were subjected to dissolution testing. Tablets exhibited greater than 50 percent dissolution at 5 minutes, greater than 70 percent at 10 minutes, greater than 85 percent at 20 minutes, and greater than 90 percent dissolution at 45 minutes after stored for 12 months at 25° C. and 60% relative humidity (RH). Tablets exhibited greater than 60 percent dissolution at 10 minutes, greater than 90 percent at 30 minutes, and greater than 95 percent dissolution at 45, 60, and 75 minutes after stored for 6 months at 45° C. and 75% relative humidity (RH).

Table 8 shows the specification of 20 mg strength tablets after stored at 25° C. and 60% relative humidity.

TABLE 8

| | Time (Month) | | | | | | |
|---|---|---|---|---|---|---|---|
| | 0 | 1 | 2 | 3 | 6 | 9 | 12 |
| Total | 0.32 | 0.24 | 0.43 | 0.19 | 0.47 | 0.48 | 0.46 |
| Impurity (%) | | | | | | | |
| Dissolution | 5 min: 47.5% | 5 min: 51.0% | 5 min: 50.4% | 5 min: 46.5% | 5 min: 52.4% | 5 min: 43.0% | 5 min. 51.0% |
| | 10 min: 71.9% | 10 min: 72.4% | 10 min. 72.6% | 10 min: 70.8% | 10 min: 73.4% | 10 min: 68.9% | 10 min: 71.7% |
| | 20 min: 87.2% | 20 min: 86.1% | 20 min: 86.1% | 20 min: 85.9% | 20 min: 86.8% | 20 min. 84.9% | 20 min: 85.5% |
| | 30 min: 91.5% | 30 min: 89.8% | 30 min: 90.7% | 30 min: 90.4% | 30 min: 90.5% | 30 min: 89.3% | 30 min: 89.1% |
| | 45 min: 92.9% | 45 min: 91.1% | 45 min: 92.0% | 45 min: 92.0% | 45 min: 91.3% | 45 min: 90.8% | 45 min: 90.2% |
| | 60 min: 93.3% | 60 min: 91.4% | 60 min: 92.0% | 60 min: 92.3% | 60 min: 91.9% | 60 min: 91.2% | 60 min: 90.7% |
| | 75 min: 93.4% | 75 min: 91.5% | 75 min: 92.2% | 75 min: 92.5% | 75 min: 92.1% | 75 min: 91.2% | 90 min: 90.7% |
| | | | | | | | 120 min: 90.7% |
| Water Content | 2.0 | 1.2 | 1.4 | 1.6 | 1.3 | 1.2 | 1.6 |
| (%) | | | | | | | |

Table 9 shows the specification of 20 mg strength tablets after stored at 40° C. and 75% relative humidity,

TABLE 9

| | Time (Month) | | | |
|---|---|---|---|---|
| | 0 | 1 | 3 | 6 |
| Total | 0.51 | 0.57 | 0.45 | 0.41 |
| Impurity | | | | |
| (%) | | | | |
| Dissolution | 5 min: 47.8% | 5 min: 37.4% | 5 min: 33.3% | 5 min: 26.8% |
| | 10 min: 70.0% | 10 min: 66.8% | 10 min: 61.5% | 10 min: 60.2% |
| | 20 min: 88.6% | 20 min: 88.0% | 20 min: 85.4% | 20 min: 84.0% |
| | 30 min: 92.6% | 30 min: 94.4% | 30 min: 92.9% | 30 min: 91.7% |
| | 45 min: 95.3% | 45 min: 97.3% | 45 min: 96.3% | 45 min: 95.2% |
| | 60 mm: 96.2% | 60 min: 98.2% | 60 min: 97.5% | 60 min: 96 4% |
| | 75 mm: 96.5% | 75 min: 98.7% | 75 min: 98.0% | 75 min: 96.7% |
| Water | 2.4 | 1.6 | 1.7 | 1.7 |
| Content | | | | |
| (%) | | | | |

### Other Embodiments

The foregoing disclosure has been described in some detail by way of illustration and example, for purposes of clarity and understanding. The invention has been described with reference to various specific and preferred embodiments and techniques. However, it should be understood that many variations and modifications can be made while remaining within the spirit and scope of the invention. It will be obvious to one of skill in the art that changes and modifications can be practiced within the scope of the appended claims. Therefore, it is to be understood that the above description is intended to be illustrative and not restrictive.

The scope of the invention should, therefore, be determined not with reference to the above description, but should instead be determined with reference to the following appended claims, along with the full scope of equivalents to which such claims are entitled.

1. A pharmaceutical composition suitable for oral administration, comprising:
a. Compound 1, which has the structure: 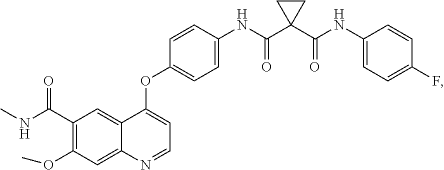 [SMILES: CNC(=O)c1cc2c(Oc3ccc(NC(=O)C4(C(=O)Nc5ccc(F)cc5)CC4)cc3)ccnc2cc1OC]
or a pharmaceutically acceptable salt thereof;
b. one or more diluents;
c. one or more binders;
d. one or more disintegrants;
e. one or more glidants;
f. one or more lubricants; and optionally
g. a film coating.

2. The pharmaceutical composition of claim 1, comprising:
a. Compound 1, or a pharmaceutically acceptable salt thereof;
b. one or more diluents selected from the group consisting an inorganic diluent, polysaccharide, mono- or disaccharide or sugar alcohol;
c. one or more binders selected from the group consisting of sodium carboxymethylcellulose, polyvinyl pyrrolidone (PVP), copovidone, polyvinyl pyrrolidone-vinyl acetate (PVP/VA) copolymer, hydroxypropylcellulose, hydroxypropyl methylcellulose and ethyl cellulose;
d. one or more disintegrants selected from the group consisting of croscarmellose sodium, crospovidone, low-substituted hydroxypropylcellulose or sodium starch glycolate;
e. one or more glidants;
f. one or more lubricants; and optionally
g. a film coating.

3. The pharmaceutical composition of claim 1 or 2, wherein the pharmaceutical composition is a capsule or tablet.

4. The pharmaceutical composition of claim 1 or 2, wherein the pharmaceutical composition is a capsule.

5. The pharmaceutical composition of claim 1 or 2, wherein the pharmaceutical composition is a tablet.

6. The pharmaceutical composition of any one of claims 1-5, containing about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, or 120 mg of Compound 1 (free base equivalent).

7. The pharmaceutical composition of any one of claims 1-5, containing at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, 99.5%, 99.9% or 100% weight/weight (w/w) of Compound 1.

8. The pharmaceutical composition of claim 2, comprising:
a. about 20 percent to about 40 percent by weight of Compound 1;
b. about 35 percent to about 45 percent by weight of microcrystalline cellulose;
c. about 15 to about 25 percent by weight of lactose;
d. about 2 to about 8 percent by weight of hydroxypropyl cellulose;
e. about 4 to about 8 percent by weight of croscarmellose sodium;
f. about 0.1 to about 0.5 percent by weight of silicon dioxide; and
g. about 0.5 to 3.5 percent by weight magnesium stearate; and optionally
h. a film coating.

9. The pharmaceutical composition of claim 2, comprising:
a. about 20 percent to about 40 percent by weight of Compound 1;
b. about 35 percent to about 45 percent by weight of microcrystalline cellulose;
c. about 15 to about 25 percent by weight of lactose;
d. about 2 to about 8 percent by weight of hydroxypropyl cellulose;
e. about 2 to about 8 percent by weight of croscarmellose sodium;
f. about 0.1 to about 0.5 percent by weight of colloidal silicon dioxide; and
g. about 1 to 5 percent by weight of stearic acid; and optionally
h. a film coating.

10. The pharmaceutical composition of claim 2, comprising:
a. about 15-150 mg of Compound 1 (free base equivalent);
b. microcrystalline cellulose;
c. lactose;
d. hydroxypropyl cellulose;
e. croscarmellose sodium;
f. silicon dioxide;
g. magnesium stearate or stearic acid; and optionally
h. a film coating.

11. The pharmaceutical composition of claim 2, comprising:
a. about 20 mg of Compound 1 (free base equivalent);
b. about 30 to about 35 mg of microcrystalline cellulose;
c. about 15 to about 18 mg anhydrous lactose;
d. about 1.5 to about 4.5 mg hydroxypropyl cellulose;
e. about 4 to about 6 mg of croscarmellose sodium;
f. about 0.1 to about 0.3 mg colloidal silicon dioxide; and
g. about 0.5 to about 0.7 mg magnesium stearate; and optionally
h. about 2 to about 6 mg of a film coating.

12. The pharmaceutical composition of claim 2, comprising:
a. about 80 mg of Compound 1 (free base equivalent);
b. about 120 to about 150 mg of microcrystalline cellulose;
c. about 60 to about 80 mg anhydrous lactose;
d. about 6 to about 18 mg hydroxypropyl cellulose;
e. about 15 to about 25 mg of croscarmellose sodium;
f. about 0.4 to about 1.5 mg colloidal silicon dioxide; and
g. about 2 to about 3 mg magnesium stearate; and optionally
h. about 8 to about 26 mg of a film coating.

13. The pharmaceutical composition of claim 2, comprising:
a. about 20 mg of Compound 1 (free base equivalent);
b. about 30 to about 40 mg of microcrystalline cellulose;
c. about 15 to about 20 mg anhydrous lactose;
d. about 3 to about 7 mg hydroxypropyl cellulose;
e. about 3 to about 7 mg of croscarmellose sodium;
f. about 0.1 to about 0.3 mg colloidal silicon dioxide; and
g. about 2 to about 4 mg stearic acid; and optionally
h. about 2 to about 5 mg of a film coating.

14. The pharmaceutical composition of claim 2, comprising:
a. about 40 mg of Compound 1 (free base equivalent);
b. about 50 to about 70 mg of microcrystalline cellulose;
c. about 25 to about 35 mg anhydrous lactose;
d. about 6 to about 10 mg hydroxypropyl cellulose;
e. about 6 to about 10 mg of croscarmellose sodium;
f. about 0.2 to about 0.6 mg colloidal silicon dioxide; and
g. about 4 to about 8 mg stearic acid; and optionally
h. about 4 to about 10 mg of a film coating.

15. The pharmaceutical composition of claim 2, comprising:
a. about 60 mg of Compound 1 (free base equivalent);
b. about 80 to about 120 mg of microcrystalline cellulose;
c. about 40 to about 70 mg anhydrous lactose;
d. about 12 to about 15 mg hydroxypropyl cellulose;
e. about 12 to about 15 mg of croscarmellose sodium;
f. about 0.5 to about 0.8 mg colloidal silicon dioxide; and
g. about 6 to about 12 mg stearic acid; and optionally
h. about 6 to about 12 mg of a film coating.

16. The pharmaceutical composition of claim 2, comprising:
a. about 80 mg of Compound 1 (free base equivalent);
b. about 120 to about 150 mg of microcrystalline cellulose;
c. about 60 to about 80 mg anhydrous lactose;
d. about 12 to about 30 mg hydroxypropyl cellulose;
e. about 12 to about 30 mg of croscarmellose sodium;
f. about 0.5 to about 1.5 mg colloidal silicon dioxide; and
g. about 8 to about 16 mg stearic acid; and optionally
h. about 8 to about 14 mg of a film coating.

17. The pharmaceutical composition of claim 2, comprising:
a. about 100 mg of Compound 1 (free base equivalent);
b. about 140 to about 160 mg of microcrystalline cellulose;
c. about 70 to about 90 mg anhydrous lactose;
d. about 15 to about 25 mg hydroxypropyl cellulose;
e. about 20 to about 30 mg of croscarmellose sodium;
f. about 0.8 to about 2.0 mg colloidal silicon dioxide; and
g. about 9 to about 18 mg stearic acid; and optionally
h. about 10 to about 30 mg of a film coating.

18. The pharmaceutical composition of claim 2, comprising:
a. about 120 mg of Compound 1 (free base equivalent);
b. about 165 to about 195 mg of microcrystalline cellulose;
c. about 80 to about 100 mg anhydrous lactose;
d. about 20 to about 30 mg hydroxypropyl cellulose;
e. about 25 to about 35 mg of croscarmellose sodium;
f. about 1.0 to about 2.5 mg colloidal silicon dioxide; and
g. about 10 to about 20 mg stearic acid; and optionally
h. about 15 to about 35 mg of a film coating.

19. The pharmaceutical composition of any one of claims 1-18, wherein Compound 1 is a crystalline (freebase) solid or a crystalline pharmaceutically acceptable salt.

20. The pharmaceutical composition of claim 19, wherein Compound 1 is a crystalline solid form selected from Form A, B, C, D, E, F, G, H, J, K, O, or Q.

21. The pharmaceutical composition of claim 19, wherein Compound 1 is a crystalline pharmaceutically acceptable salt selected from the group consisting of Compound 1 HCl salt, Compound 1 fumaric acid salt, and Compound 1 phosphoric acid salt.

22. The pharmaceutical composition of claim 21, wherein the Compound 1 HCl salt is selected from the crystalline salt forms Compound 1 HCl salt Forms A, B, C, and D.

23. The pharmaceutical composition of claim 21, wherein the Compound 1 fumaric acid salt is selected from the crystalline salt forms Compound 1 fumaric acid Form A and Compound 1 hemifumarate Form B.

24. The pharmaceutical composition of claim 23, wherein the Compound 1 fumaric acid salt is Compound 1 hemifumarate Form B.

25. The pharmaceutical composition of claim 21, wherein the Compound 1 phosphoric acid salt form is crystalline Compound 1 phosphoric acid salt form A.

26. A method of treating a proliferative disease or disorder in a patient in need of such treatment, comprising administering to the patient the pharmaceutical composition of any one of claims 1-25.

27. The method of claim 26, wherein the proliferative disease or disorder is cancer.

28. A process of making a tablet of the pharmaceutical composition of any one of claims 1-3 and 5-25, comprising de-lumping excipients; granulating a mixture comprising Compound 1, the excipients and water through high-shear granulation to produce wet granules; de-lumping the wet granules; drying the wet granules through a fluid bed dryer to afford dried granules; milling the dried granules to afford milled granules; blending the milled granules with a disintegrant and/or a glidant to afford blended granules; lubricating the blended granules by blending the blended granules with a lubricant to produce a lubricated blend; and compressing the lubricated blend to form the tablet.

29. The process of claim 28, further comprising coating the tablet.

30. The process of claim 28 or 29, wherein the disintegrants and/or the glidants are croscarmellose sodium and colloidal silicon dioxide.

31. The process of any of claims 28-30, wherein the lubricant is magnesium stearate.

32. The process of any of claims 28-30, wherein the lubricant is stearic acid.
